(19) United States
(12) Patent Application Publication
(10) Pub. No.: US 2025/0213820 A1
(43) Pub. Date: Jul. 3, 2025

(54) METHOD FOR PROVIDING USER INTERFACE FOR CONTROLLING MAGNETIC CATHETER BY CHANGING EXTERNAL MAGNETIC FIELD, AND DEVICE FOR PROVIDING USER INTERFACE USING SAME

(71) Applicants: SEOUL NATIONAL UNIVERSITY HOSPITAL, Seoul (KR); SEOUL NATIONAL UNIVERSITY R&DB FOUNDATION, Seoul (KR)

(72) Inventors: Chang Hwan YOON, Seoul (KR); Jung Ik HA, Seoul (KR); Sang Won LEE, Seoul (KR)

(21) Appl. No.: 18/851,309

(22) PCT Filed: Feb. 6, 2023
(86) PCT No.: PCT/KR2023/001651

§ 371 Date: Sep. 26, 2024

(30) Foreign Application Priority Data
Mar. 30, 2022 (KR) 10-2022-0039979

(51) Int. Cl. A61M 25/01

(52) U.S. Cl. CPC A61M 25/0127; A61M 2205/3313; A61M 2205/502; A61M 2205/70

(57) ABSTRACT

A method for providing a user interface for controlling a magnetic catheter by changing an external magnetic field, including steps of: acquiring a k-th subject spatial image by photographing a subject space from a k-th subject spatial absolute coordinate point, displaying a k-th selection indicating symbol at a k-th subject spatial relative coordinate point, which is a relative coordinate point selected by a user with respect to the k-th subject spatial absolute coordinate point as its origin, displaying a k-th visualized coordinate space to expose a yaw and a pitch of a current direction of the magnetic catheter, and continuously exposing the yaw and the pitch of the current direction of the magnetic catheter as time passes.

### FIELD OF THE DISCLOSURE

The present disclosure relates to a method for providing a user interface capable of controlling a magnetic catheter by changing an external magnetic field and a user interface providing device using the same; and more particularly, the method for providing the user interface by visualizing a direction of the external magnetic field generated and also visualizing an updated direction of the external magnetic field after changing the external magnetic field, to thereby effectively control the magnetic catheter, and the user interface providing device using the same.

### BACKGROUND OF THE DISCLOSURE

A catheter is a thin tube made from medical grade materials that can be inserted into a patient's cavity, duct, or vessel during a surgical procedure.

Since it is not possible to see what is actually going on when the catheter is inside the patient, a visualizing tool is required for a practitioner to view the surgical procedure. However, the visualizing tool has a limitation of only providing one-way information to the practitioner.

According a prior art disclosed in Korean Registered Patent No. 10-2245665, a method of providing a position of a magnetic catheter together with an ultrasound image (taken by a camera module mounted on the magnetic catheter) of internals of the patient, to thereby allow the practitioner to accurately view the internals of the patient is disclosed.

However, the prior art merely provides the ultrasound image and the position of the magnetic catheter on a display for the practitioner to refer thereto and does not disclose a system that can interact with the practitioner to support a precise control of the magnetic catheter.

Therefore, it is necessary to achieve a method for providing a user interface through which the practitioner can interact with the system, thereby allowing the practitioner to directly control the magnetic catheter by using an external magnetic field.

### SUMMARY OF THE DISCLOSURE

It is an object of the present disclosure to solve all the aforementioned problems.

It is another object of the present disclosure to provide a user interface that can interact with a user to allow the user to check a position of a magnetic catheter and to control a change in a direction of the magnetic catheter by controlling a direction of an external magnetic field.

It is still another object of the present disclosure to provide the user interface that can continuously display information on a direction of the external magnetic field used for changing the direction of the magnetic catheter.

It is still yet another object of the present disclosure to provide the user interface capable of allowing a magnetic field generating device to accurately calculate each of required currents to be applied to each of coils in order to generate the external magnetic field used for changing the direction of the magnetic catheter.

In order to accomplish objects above, representative structures of the present disclosure are described as follows: In accordance with one aspect of the present disclosure, there is provided a method for providing a user interface, including steps of: (a) in response to acquiring a k-th subject spatial image, which is obtained by photographing a part of subject space from a k-th subject spatial absolute coordinate point through a subject spatial image managing device connected to a user interface providing device, wherein k is an integer greater than or equal to 1, and wherein the subject space is affected by an external magnetic field, (i) displaying, by the user interface providing device, the k-th subject spatial image on a 1-st region of a display of the user interface providing device, and (ii) in response to acquiring selection information of a k-th subject spatial relative coordinate point, which is a relative coordinate point with respect to the k-th subject spatial absolute coordinate point, corresponding to a position of a magnetic catheter positioned within the subject space, displaying, by the user interface providing device, a k-th selection indicating symbol at a position corresponding to the k-th subject spatial relative coordinate point in the k-th subject spatial image; and (b) on condition that a (T_k_1)-st time point represents a time point when the k-th subject spatial relative coordinate point is selected, and each of a (T_k_2)-nd time point to a (T_k_p)-th time point represents each time point when the external magnetic field is applied upon or a change of the external magnetic field is made to the k-th subject spatial relative coordinate point, wherein the (T_k_1)-st time point to the (T_k_p)-th time point are within a (T_k)-th time range that starts from a time point of acquiring the k-th subject spatial image to a time point of acquiring a (k+1)-th subject spatial image by re-photographing a part of the subject space after a change in a position of the magnetic catheter is made, with p being an integer greater than or equal to 2, (b1) (i) setting, by the user interface providing device, a k-th sub coordinate space having each of coordinate axes corresponding to the k-th subject spatial image while using the k-th subject spatial relative coordinate point as its origin by referring to the k-th subject spatial image and the k-th subject spatial relative coordinate point, and (ii) acquiring, by the user interface providing device, information on a (T_k_1)-st yaw and a (T_k_1)-st pitch representing direction information of where the magnetic catheter is facing at the (T_k_1)-st time point with respect to a k-th reference coordinate axis, which is a specific coordinate axis among coordinate axes corresponding to the k-th sub coordinate space, (b2) (i) displaying, by the user interface providing device, a k-th visualized coordinate space, which is acquired by visualizing the k-th sub coordinate space, on a (2_1)-st region of the display, and (ii) displaying, by the user interface providing device, a (T_k)-th current graphical element to be exposed in a direction corresponding to the (T_k_1)-st yaw and the (T_k_1)-st pitch with respect to an origin of the k-th visualized coordinate space; and (b3) in response to confirming, during a time section of after a (T_k_(q−1))-th time point and before a (T_k_q)-th time point, (1) information on a (T_k_q)-th magnetic field to be generated at the (T_k_q)-th time point with respect to the k-th subject spatial relative coordinate point, and (2) an instruction to generate the (T_k_q)-th magnetic field, wherein the information on the (T_k_q)-th magnetic field includes a (T_k_q)-th yaw, a (T_k_q)-th pitch, and a (T_k_q)-th desired magnetic field strength of the (T_k_q)-th magnetic field, and wherein the (T_k_q)-th time point is a specific time point within a time range starting from the (T_k_2)-nd time point to the (T_k_p)-th time point, with q being an integer greater than or equal to 2 and less than or equal to p, (i) at the (T_k_q)-th time point, changing, by the user interface providing device, the (T_k)-th current graphical element, which has been exposed in a direction of a (T_k_(q−1))-th yaw and a (T_k_(q−1))-th pitch corresponding to the (T_k_(q−1))-th time point, to be exposed in a direction of the (T_k_q)-th yaw and the (T_k_q)-th pitch corresponding to the (T_k_q)-th time point, by referring to the (T_k_q)-th yaw, the (T_k_q)-th pitch, and the (T_k_q)-th desired magnetic field strength, (ii) updating, by the user interface providing device, each of the (T_k_q)-th pitch to the (T_k_p)-th pitch, to thereby display thereof on a (2_2)-nd region of the display, and (iii) continuously displaying, by the user interface providing device, the (T_k_q)-th desired magnetic field strength on a (2_3)-rd region of the display starting from the (T_k_2)-nd time point to the (T_k_p)-th time point.

As one example, after the step of (b), the method further includes steps of: (c) after a generation of a (T_k_p)-th magnetic field at the (T_k_p)-th time point with respect to the k-th subject spatial relative coordinate point, in response to acquiring the (k+1)-th subject spatial image by re-photographing a part of the subject space due to a change in the position of the magnetic catheter through the subject spatial image managing device, (i) displaying, by the user interface providing device, the (k+1)-th subject spatial image on the 1-st region of the display, and (ii) in response to acquiring selection information of a (k+1)-th subject spatial relative coordinate point corresponding to a changed position of the magnetic catheter, displaying, by the user interface providing device, a (k+1)-th selection indicating symbol at a position corresponding to the (k+1)-th subject spatial relative coordinate point on the (k+1)-th subject spatial image, wherein, in case the (k+1)-th subject spatial image is re-photographed from the k-th subject spatial absolute coordinate point, the (k+1)-th subject spatial relative coordinate point is a relative coordinate point with respect to the k-th subject spatial absolute coordinate point and wherein, in case the (k+1)-th subject spatial image is re-photographed from a (k+1)-th subject spatial absolute coordinate point that is different from the k-th subject spatial absolute coordinate point, the (k+1)-th subject spatial relative coordinate point is a relative coordinate point with respect to the (k+1)-th subject spatial absolute coordinate point; and (d) on condition that a (T_(k+1)_1)-st time point represents a time point when the (k+1)-th subject spatial relative coordinate point is selected, and each of a (T_(k+1)_2)-nd time point to a (T_(k+1)_m)-th time point represents each time point when the external magnetic field is applied upon or a change of the external magnetic field is made to the (k+1)-th subject spatial relative coordinate point, wherein the (T (k+1)_1)-st time point to the (T (k+1) m)-th time point is within a (T (k+1))-th time range that starts from a time point of acquiring the (k+1)-th subject spatial image to a time point of acquiring a (k+2)-th subject spatial image by re-photographing a part of the subject space after a change in a position of the magnetic catheter is made, with m being an integer greater than or equal to 2, (d1) (i) setting, by the user interface providing device, a (k+1)-th sub coordinate space having each of coordinate axes corresponding to the (k+1)-th subject spatial image while using the (k+1)-th subject spatial relative coordinate point as its origin by referring to the (k+1)-th subject spatial image and the (k+1)-th subject spatial relative coordinate point, and (ii) acquiring, by the user interface providing device, information on a (T (k+1)_1)-st yaw and a (T (k+1)_1)-st pitch representing direction information of where the magnetic catheter is facing at the (T (k+1)_1)-st time point with respect to a (k+1)-th reference coordinate axis, which is a specific coordinate axis among coordinate axes corresponding to the (k+1)-th sub coordinate space, (d2) (i) displaying, by the user interface providing device, a (k+1)-th visualized coordinate space, which is acquired by visualizing the (k+1)-th sub coordinate space, on the (2_1)-st region of the display, and (ii) displaying, by the user interface providing device, a (T_(k+1))-th current graphical element to be exposed in a direction corresponding to the (T_(k+1)_1)-st yaw and the (T_(k+1)_1)-st pitch with respect to an origin of the (k+1)-th visualized coordinate space; and (d3) in response to confirming, during a time section of after a (T_(k+1)_(n−1))-th time point and before a (T_(k+1)_n)-th time point, (1) information on a (T_(k+1)_n)-th magnetic field to be generated at the (T_(k+1)_n)-th time point with respect to the (k+1)-th subject spatial relative coordinate point, and (2) an instruction to generate the (T_(k+1)_n)-th magnetic field, wherein the information on the (T_(k+1)_n)-th magnetic field includes a (T_(k+1)_n)-th yaw, a (T_(k+1)_n)-th pitch, and a (T_(k+1)_n)-th desired magnetic field strength of the (T_(k+1)_n)-th magnetic field, and wherein the (T_(k+1)_n)-th time point is a specific time point within a time range starting from the (T_(k+1)_2)-nd time point to the (T_(k+1)_m)-th time point, with n being an integer greater than or equal to 2 and less than or equal to m, (i) at the (T_(k+1)_n)-th time point, changing, by the user interface providing device, the (T_(k+1))-th current graphical element, which has been exposed in a direction of a (T_(k+1)_(n−1))-th yaw and a (T_(k+1)_(n−1))-th pitch corresponding to the (T_(k+1)_(n−1))-th time point, to be exposed in a direction of the (T_(k+1)_n)-th yaw and the (T_(k+1)_n)-th pitch corresponding to the (T_(k+1)_n)-th time point, by referring to the (T_(k+1)_n)-th yaw, the (T_(k+1)_n)-th pitch, and the (T_(k+1)_n)-th desired magnetic field strength, (ii) updating, by the user interface providing device, each of the (T_(k+1)_n)-th pitch to the (T_(k+1) m)-th pitch, to thereby display thereof on the (2_2)-nd region of the display, and (iii) continuously displaying, by the user interface providing device, the (T_(k+1)_n)-th desired magnetic field strength on the (2_3)-rd region of the display starting from the (T_(k+1)_2)-nd time point to the (T_(k+1) m)-th time point.

As one example, at the step of (b1), the user interface providing device acquires target direction information, including a (T_k_p)-th yaw and a (T_k_p)-th pitch, corresponding to the (T_k_p)-th magnetic field to be generated at the (T_k_p)-th time point on the k-th subject spatial relative coordinate point, to thereby allow, at the step of (b2), (i) a (T_k)-th starting graphical element to be exposed in a direction corresponding to the (T_k_1)-st yaw and the (T_k_1)-st pitch with respect to the origin of the k-th visualized coordinate space, and (ii) a (T_k)-th target graphical element to be exposed in a direction corresponding to the (T_k_p)-th yaw and the (T_k_p)-th pitch with respect to the origin of the k-th visualized coordinate space; and wherein, at the step of (d1), the user interface providing device acquires target direction information, including a (T_(k+1)_m)-th yaw and a (T_(k+1)_m)-th pitch, corresponding to a (T_(k+1) m)-th magnetic field to be generated at the (T_(k+1) m)-th time point on the (k+1)-th subject spatial relative coordinate point, to thereby allow, at the step of (d2), (i) a (T_(k+1))-th starting graphical element to be exposed in a direction corresponding to a (T_(k+1)_1)-st yaw and a (T_(k+1)_1)-st pitch with respect to the origin of the (k+1)-th visualized coordinate space, and (ii) a (T_(k+1))-th target graphical element to be exposed in a direction corresponding to the (T_(k+1)_m)-th yaw and the (T_(k+1)_m)-th pitch with respect to the origin of the (k+1)-th visualized coordinate space.

As one example, at the step of (d1), the user interface providing device sets the (T_(k+1)_1)-st yaw and the (T_(k+1)_1)-st pitch representing direction information of where the magnetic catheter is facing at the (T_(k+1)_1)-st time point with respect to the (k+1)-th reference coordinate axis to be same as a direction corresponding to the (T_k_p)-th yaw and the (T_k_p)-th pitch with respect to the k-th reference coordinate axis, by referring to (1) the (T_k_p)-th yaw and the (T_k_p)-th pitch corresponding to the (T_k_p)-th magnetic field generated at the (T_k_p)-th time point on the k-th subject spatial relative coordinate point and (2) the k-th reference coordinate axis.

As one example, at the step of (a), the user interface providing device (1) sets a (k_1)-st direction corresponding to a horizontal direction of the k-th subject spatial image as an image x-axis, a (k_2)-nd direction that is perpendicular to the (k_1)-st direction of the k-th subject spatial image as an image y-axis, and a direction that is perpendicular to a plane formed by the image x-axis and the image y-axis as an image z-axis, and (2) displays the k-th selection indicating symbol corresponding to the k-th subject spatial relative coordinate point on the k-th subject spatial image, wherein at least a part of a color, a brightness, and a chroma of the k-th selection indicating symbol is determined to vary according to a predetermined condition depending on an image z-coordinate of the k-th subject spatial relative coordinate point that represents a z-coordinate value corresponding to the k-th subject spatial relative coordinate point on the image z-axis; and wherein, at the step of (c), the user interface providing device (3) updates a ((k+1)_1)-st direction corresponding to a horizontal direction of the (k+1)-th subject spatial image to be the image x-axis, updates a ((k+1)_2)-nd direction corresponding to a vertical direction of the (k+1)-th subject spatial image that is perpendicular to the ((k+1)−1)-st direction to be the image y-axis, and updates a direction that is perpendicular to the plane formed by the updated image x-axis and the updated image y-axis to be the image z-axis, and (4) displays the (k+1)-th selection indicating symbol corresponding to the (k+1)-th subject spatial relative coordinate point on the (k+1)-th subject spatial image, wherein at least a part of color, a brightness, and a chroma of the (k+1)-th selection indicating symbol is determined to vary according to an image z-coordinate of the (k+1)-th subject spatial relative coordinate point that represents a z-coordinate value corresponding to the (k+1)-th subject spatial relative coordinate point on the updated image z-axis.

As one example, each of the k-th visualized coordinate space and the (k+1)-th visualized coordinate space is a 3-dimensional space including a visualized x-axis, a visualized y-axis perpendicular to the visualized x-axis, and a visualized z-axis perpendicular to a plane formed by the visualized x-axis and the visualized y-axis, wherein each of the visualized x-axis, the visualized y-axis, and the visualized z-axis is set to correspond to the image x-axis, the image y-axis, and the image z-axis, and wherein (1) each of a (T_k)-th starting graphical element, the (T_k)-th current graphical element, and a (T_k)-th target graphical element is allowed to be displayed on the k-th visualized coordinate space, and (2) each of a (T_(k+1))-th starting graphical element, the (T_(k+1))-th current graphical element, and a (T_(k+1))-th target graphical element is allowed to be displayed on the (k+1)-th visualized coordinate space, wherein at least one of a color, a brightness, and a chroma of each of the (T_k)-th starting graphical element, the (T_k)-th current graphical element, the (T_k)-th target graphical element, the (T_(k+1))-th starting graphical element, the (T_(k+1))-th current graphical element, and the (T_(k+1))-th target graphical element is determined to vary according to each value of each visualized z-coordinate thereof that represents each coordinate value of a z-coordinate corresponding thereto on the visualized z-axis.

As one example, at the step of (a), the user interface providing device allows (i) k-th relative coordinates acquired by calculating coordinate values of the k-th subject spatial relative coordinate point while using the k-th subject spatial absolute coordinate point as its origin and (ii) k-th absolute coordinates acquired by converting coordinate values of the k-th relative coordinates within the subject space, to be displayed on at least part of the 1-st region of the display or on a 3-rd region of the display; and wherein at the step of (c), the user interface providing device allows (i) (k+1)-th relative coordinates acquired by calculating coordinate values of the (k+1)-th subject spatial relative coordinate point while using the (k+1)-th subject spatial absolute coordinate point as its origin and (ii) (k+1)-th absolute coordinates acquired by converting coordinate values of the (k+1)-th relative coordinates within the subject space, to be displayed on at least part of the 1-st region of the display or on the 3-rd region of the display by updating previous coordinates.

As one example, the external magnetic field is generated by a coil system connected to the user interface providing device, wherein the coil system includes a 1-st coil to a j-th coil, wherein each of the 1-st coil to the j-th coil is applied with each of a 1-st current to a j-th current, to thereby generate each of a 1-st sub-magnetic field to a j-th sub-magnetic field, wherein the external magnetic field is generated by overlapping each of the 1-st sub-magnetic field to the j-th sub-magnetic field; and wherein (i) each of the 1-st current to the j-th current to be applied to each of the 1-st coil the j-th coil at the (T_k_q)-th time point is determined by referring to the (T_k_q)-th yaw, the (T_k_p)-th pitch, and the (T_k_q)-th desired magnetic field strength corresponding to the instruction to generate the (T_k_q)-th magnetic field and (ii) each of the 1-st current to the j-th current to be applied to each of the 1-st coil the j-th coil at the (T_(k+1) m)-th time point is determined by referring to the (T_(k+1)_m)-th yaw, the (T_(k+1)_m)-th pitch, and the (T_(k+1)_m)-th desired magnetic field corresponding to the instruction to generate the (T_(k+1)_m)-th magnetic field.

As one example, on condition that the user interface providing device has stored each information on a measured magnetic flux density, which is measured in advance for each of specific coordinate points that are a plurality of points among all coordinate points of the subject space, in each of magnetic flux density tables corresponding to the specific coordinate points when a certain magnetic field as the external magnetic field is generated by applying a certain current as each of the 1-st current to the j-th current to each of the 1-st coil to the j-th coil, each of the magnetic flux density tables corresponding to a point P is represented as an array A(P), wherein the point P is one of the specific coordinate points and is the k-th subject spatial relative coordinate point or the (k+1)-th subject spatial relative coordinate point, wherein

$$A ( P ) = [ \begin{matrix}{A}_{x 1} ( P ) & {A}_{x 2} ( P ) & … & {A}_{xj} ( P ) \\ {A}_{y 1} ( P ) & {A}_{y 2} ( P ) & … & {A}_{y j} ( P ) \\ {A}_{z 1} ( P ) & {A}_{z 2} ( P ) & … & {A}_{z j} ( P )\end{matrix} ] ,$$

wherein the array A(P) includes Aₓ₁(P) to Aₓⱼ(P), A_{y1}(P) to A_{yj}(P), and A_{z1}(P) to A_{zj}(P), wherein Aₓⱼ(P) represents a (j_1)-st magnetic flux density of the j-th sub-magnetic field generated by the j-th coil in a first direction as an subject space x-axis with respect to the point P, A_{yj}(P) represents a (j_2)-nd magnetic flux density of the j-th sub-magnetic field generated by the j-th coil in a second direction as an subject space y-axis with respect to the point P, and A_{zj}(P) represents a (j_3)-rd magnetic flux density of the j-th sub-magnetic field generated by the j-th coil in a third direction as a direction perpendicular to a plane formed by the subject space x-axis and the subject space y-axis with respect to the point P; and wherein B_{ref}(P) represents the instruction to generate the (T_k_q)-th magnetic field or the instruction to generate the (T_(k+1)_m)-th magnetic field with respect to the point P, wherein the B_{ref}(P) and A(P)I* follow a relationship as below:

$${B}_{ref} ( P ) = [ \begin{matrix}{A}_{x 1} ( P ) & {A}_{x 2} ( P ) & … & {A}_{xj} ( P ) \\ {A}_{y 1} ( P ) & {A}_{y 2} ( P ) & … & {A}_{y j} ( P ) \\ {A}_{z 1} ( P ) & {A}_{z 2} ( P ) & … & {A}_{z j} ( P )\end{matrix} ] [ \begin{matrix}{i}_{1}^{*} \\ ⋮ \\ {i}_{j}^{*}\end{matrix} ] = A ( P ) {I}^{*}$$

wherein, I* is an array whose components include values of each of the 1-st current to the j-th current that is applied to each of the 1-st coil to the j-th coil, wherein each of ij to ij represents values of each of the 1-st current to the j-th current, and wherein the 1-st current to the j-th current follow a formula below:

$${I}^{*} = [ \begin{matrix}{i}_{1}^{*} \\ ⋮ \\ {i}_{j}^{*}\end{matrix} ] = {A ( A {A}^{T} )}^{- 1} {B}_{r e f} ( P ) .$$

As one example, on condition that the k-th subject spatial relative coordinate point or the (k+1)-th subject spatial relative coordinate point is not one of the specific coordinate points, the user interface providing device performs a predetermined calibration, which includes a linear interpolation calibration, (i) on a magnetic flux density table corresponding to the k-th subject spatial relative coordinate point by referring to each of k-th specific magnetic flux density tables corresponding to at least two or more k-th specific coordinate points, which are within a predetermined distance from the k-th subject spatial relative coordinate point or (ii) on a magnetic flux density table corresponding to the (k+1)-th subject spatial relative coordinate point by referring to each of (k+1)-th specific magnetic flux density tables corresponding to at least two or more (k+1)-th specific coordinate points, which are within a predetermined distance from the (k+1)-th subject spatial relative coordinate point.

In accordance with another aspect of the present disclosure, there is provided a user interface providing device for providing a user interface, including: at least one memory that stores instructions; at least one processor configured to execute the instructions to perform processes of: (I) in response to acquiring a k-th subject spatial image, which is obtained by photographing a part of subject space from a k-th subject spatial absolute coordinate point through a subject spatial image managing device connected to the user interface providing device, wherein k is an integer greater than or equal to 1, and wherein the subject space is affected by an external magnetic field, (i) displaying the k-th subject spatial image on a 1-st region of a display of the user interface providing device, and (ii) in response to acquiring selection information of a k-th subject spatial relative coordinate point, which is a relative coordinate point with respect to the k-th subject spatial absolute coordinate point, corresponding to a position of a magnetic catheter positioned within the subject space, displaying a k-th selection indicating symbol at a position corresponding to the k-th subject spatial relative coordinate point in the k-th subject spatial image; and (II) on condition that a (T_k_1)-st time point represents a time point when the k-th subject spatial relative coordinate point is selected, and each of a (T_k_2)-nd time point to a (T_k_p)-th time point represents each time point when the external magnetic field is applied upon or a change of the external magnetic field is made to the k-th subject spatial relative coordinate point, wherein the (T_k_1)-st time point to the (T_k_p)-th time point are within a (T_k)-th time range that starts from a time point of acquiring the k-th subject spatial image to a time point of acquiring a (k+1)-th subject spatial image by re-photographing a part of the subject space after a change in a position of the magnetic catheter is made, with p being an integer greater than or equal to 2, (II-1) (i) setting a k-th sub coordinate space having each of coordinate axes corresponding to the k-th subject spatial image while using the k-th subject spatial relative coordinate point as its origin by referring to the k-th subject spatial image and the k-th subject spatial relative coordinate point, and (ii) acquiring information on a (T_k_1)-st yaw and a (T_k_1)-st pitch representing direction information of where the magnetic catheter is facing at the (T_k_1)-st time point with respect to a k-th reference coordinate axis, which is a specific coordinate axis among coordinate axes corresponding to the k-th sub coordinate space, (II-2) (i) displaying a k-th visualized coordinate space, which is acquired by visualizing the k-th sub coordinate space, on a (2_1)-st region of the display, and (ii) displaying a (T_k)-th current graphical element to be exposed in a direction corresponding to the (T_k_1)-st yaw and the (T_k_1)-st pitch with respect to an origin of the k-th visualized coordinate space; and (II-3) in response to confirming, during a time section of after a (T_k_(q−1))-th time point and before a (T_k_q)-th time point, (1) information on a (T_k_q)-th magnetic field to be generated at the (T_k_q)-th time point with respect to the k-th subject spatial relative coordinate point, and (2) an instruction to generate the (T_k_q)-th magnetic field, wherein the information on the (T_k_q)-th magnetic field includes a (T_k_q)-th yaw, a (T_k_q)-th pitch, and a (T_k_q)-th desired magnetic field strength of the (T_k_q)-th magnetic field, and wherein the (T_k_q)-th time point is a specific time point within a time range starting from the (T_k_2)-nd time point to the (T_k_p)-th time point, with q being an integer greater than or equal to 2 and less than or equal to p, (i) at the (T_k_q)-th time point, changing the (T_k)-th current graphical element, which has been exposed in a direction of a (T_k_(q−1))-th yaw and a (T_k_(q−1))-th pitch corresponding to the (T_k_(q−1))-th time point, to be exposed in a direction of the (T_k_q)-th yaw and the (T_k_q)-th pitch corresponding to the (T_k_q)-th time point, by referring to the (T_k_q)-th yaw, the (T_k_q)-th pitch, and the (T_k_q)-th desired magnetic field strength, (ii) updating each of the (T_k_q)-th pitch to the (T_k_p)-th pitch, to thereby display thereof on a (2_2)-nd region of the display, and (iii) continuously displaying the (T_k_q)-th desired magnetic field strength on a (2_3)-rd region of the display starting from the (T_k_2)-nd time point to the (T_k_p)-th time point.

As one example, after the process of (II), the processor further performs processes of: (III) after a generation of a (T_k_p)-th magnetic field at the (T_k_p)-th time point with respect to the k-th subject spatial relative coordinate point, in response to acquiring the (k+1)-th subject spatial image by re-photographing a part of the subject space due to a change in the position of the magnetic catheter through the subject spatial image managing device, (i) displaying the (k+1)-th subject spatial image on the 1-st region of the display, and (ii) in response to acquiring selection information of a (k+1)-th subject spatial relative coordinate point corresponding to a changed position of the magnetic catheter, displaying a (k+1)-th selection indicating symbol at a position corresponding to the (k+1)-th subject spatial relative coordinate point on the (k+1)-th subject spatial image, wherein, in case the (k+1)-th subject spatial image is re-photographed from the k-th subject spatial absolute coordinate point, the (k+1)-th subject spatial relative coordinate point is a relative coordinate point with respect to the k-th subject spatial absolute coordinate point and wherein, in case the (k+1)-th subject spatial image is re-photographed from a (k+1)-th subject spatial absolute coordinate point that is different from the k-th subject spatial absolute coordinate point, the (k+1)-th subject spatial relative coordinate point is a relative coordinate point with respect to the (k+1)-th subject spatial absolute coordinate point; and (IV) on condition that a (T_(k+1)_1)-st time point represents a time point when the (k+1)-th subject spatial relative coordinate point is selected, and each of a (T_(k+1)_2)-nd time point to a (T_(k+1) m)-th time point represents each time point when the external magnetic field is applied upon or a change of the external magnetic field is made to the (k+1)-th subject spatial relative coordinate point, wherein the (T_(k+1)_1)-st time point to the (T_(k+1) m)-th time point is within a (T_(k+1))-th time range that starts from a time point of acquiring the (k+1)-th subject spatial image to a time point of acquiring a (k+2)-th subject spatial image by re-photographing a part of the subject space after a change in a position of the magnetic catheter is made, with m being an integer greater than or equal to 2, (IV-1) (i) setting a (k+1)-th sub coordinate space having each of coordinate axes corresponding to the (k+1)-th subject spatial image while using the (k+1)-th subject spatial relative coordinate point as its origin by referring to the (k+1)-th subject spatial image and the (k+1)-th subject spatial relative coordinate point, and (ii) acquiring information on a (T_(k+1)_1)-st yaw and a (T_(k+1)_1)-st pitch representing direction information of where the magnetic catheter is facing at the (T_(k+1)_1)-st time point with respect to a (k+1)-th reference coordinate axis, which is a specific coordinate axis among coordinate axes corresponding to the (k+1)-th sub coordinate space, (IV-2) (i) displaying a (k+1)-th visualized coordinate space, which is acquired by visualizing the (k+1)-th sub coordinate space, on the (2_1)-st region of the display, and (ii) displaying a (T_(k+1))-th current graphical element to be exposed in a direction corresponding to the (T_(k+1)_1)-st yaw and the (T_(k+1)_1)-st pitch with respect to an origin of the (k+1)-th visualized coordinate space; and (IV-3) in response to confirming, during a time section of after a (T_(k+1)_(n−1))-th time point and before a (T_(k+1)_n)-th time point, (1) information on a (T_(k+1)_n)-th magnetic field to be generated at the (T_(k+1)_n)-th time point with respect to the (k+1)-th subject spatial relative coordinate point, and (2) an instruction to generate the (T_(k+1)_n)-th magnetic field, wherein the information on the (T_(k+1)_n)-th magnetic field includes a (T_(k+1)_n)-th yaw, a (T_(k+1)_n)-th pitch, and a (T_(k+1)_n)-th desired magnetic field strength of the (T_(k+1)_n)-th magnetic field, and wherein the (T_(k+1)_n)-th time point is a specific time point within a time range starting from the (T_(k+1)_2)-nd time point to the (T_(k+1)_m)-th time point, with n being an integer greater than or equal to 2 and less than or equal to m, (i) at the (T_(k+1)_n)-th time point, changing the (T_(k+1))-th current graphical element, which has been exposed in a direction of a (T_(k+1)_(n−1))-th yaw and a (T_(k+1)_(n−1))-th pitch corresponding to the (T_(k+1)_(n−1))-th time point, to be exposed in a direction of the (T_(k+1)_n)-th yaw and the (T_(k+1)_n)-th pitch corresponding to the (T_(k+1)_n)-th time point, by referring to the (T_(k+1)_n)-th yaw, the (T_(k+1)_n)-th pitch, and the (T_(k+1)_n)-th desired magnetic field strength, (ii) updating each of the (T_(k+1)_n)-th pitch to the (T_(k+1)_m)-th pitch, to thereby display thereof on the (2_2)-nd region of the display, and (iii) continuously displaying the (T_(k+1)_n)-th desired magnetic field strength on the (2_3)-rd region of the display starting from the (T_(k+1)_2)-nd time point to the (T_(k+1)_m)-th time point.

As one example, at the process of (II-1), the processor acquires target direction information, including a (T_k_p)-th yaw and a (T_k_p)-th pitch, corresponding to the (T_k_p)-th magnetic field to be generated at the (T_k_p)-th time point on the k-th subject spatial relative coordinate point, to thereby allow, at the process of (II-2), (i) a (T_k)-th starting graphical element to be exposed in a direction corresponding to the (T_k_1)-st yaw and the (T_k_1)-st pitch with respect to the origin of the k-th visualized coordinate space, and (ii) a (T_k)-th target graphical element to be exposed in a direction corresponding to the (T_k_p)-th yaw and the (T_k_p)-th pitch with respect to the origin of the k-th visualized coordinate space; and wherein, at the process of (IV-1), the processor acquires target direction information, including a (T_(k+1)_m)-th yaw and a (T_(k+1)_m)-th pitch, corresponding to a (T_(k+1)_m)-th magnetic field to be generated at the (T_(k+1) m)-th time point on the (k+1)-th subject spatial relative coordinate point, to thereby allow, at the process of (IV-2), (i) a (T_(k+1))-th starting graphical element to be exposed in a direction corresponding to a (T_(k+1)_1)-st yaw and a (T_(k+1)_1)-st pitch with respect to the origin of the (k+1)-th visualized coordinate space, and (ii) a (T_(k+1))-th target graphical element to be exposed in a direction corresponding to the (T_(k+1)_m)-th yaw and the (T_(k+1)_m)-th pitch with respect to the origin of the (k+1)-th visualized coordinate space.

As one example, at the process of (IV-1), the processor sets the (T_(k+1)_1)-st yaw and the (T_(k+1)_1)-st pitch representing direction information of where the magnetic catheter is facing at the (T_(k+1)_1)-st time point with respect to the (k+1)-th reference coordinate axis to be same as a direction corresponding to the (T_k_p)-th yaw and the (T_k_p)-th pitch with respect to the k-th reference coordinate axis, by referring to (1) the (T_k_p)-th yaw and the (T_k_p)-th pitch corresponding to the (T_k_p)-th magnetic field generated at the (T_k_p)-th time point on the k-th subject spatial relative coordinate point and (2) the k-th reference coordinate axis.

As one example, at the process of (I), the processor (1) sets a (k_1)-st direction corresponding to a horizontal direction of the k-th subject spatial image as an image x-axis, a (k_2)-nd direction that is perpendicular to the (k_1)-st direction of the k-th subject spatial image as an image y-axis, and a direction that is perpendicular to a plane formed by the image x-axis and the image y-axis as an image z-axis, and (2) displays the k-th selection indicating symbol corresponding to the k-th subject spatial relative coordinate point on the k-th subject spatial image, wherein at least a part of a color, a brightness, and a chroma of the k-th selection indicating symbol is determined to vary according to a predetermined condition depending on an image z-coordinate of the k-th subject spatial relative coordinate point that represents a z-coordinate value corresponding to the k-th subject spatial relative coordinate point on the image z-axis; and wherein, at the process of (III), the processor (3) updates a ((k+1)_1)-st direction corresponding to a horizontal direction of the (k+1)-th subject spatial image to be the image x-axis, updates a ((k+1)_2)-nd direction corresponding to a vertical direction of the (k+1)-th subject spatial image that is perpendicular to the ((k+1)−1)-st direction to be the image y-axis, and updates a direction that is perpendicular to the plane formed by the updated image x-axis and the updated image y-axis to be the image z-axis, and (4) displays the (k+1)-th selection indicating symbol corresponding to the (k+1)-th subject spatial relative coordinate point on the (k+1)-th subject spatial image, wherein at least a part of color, a brightness, and a chroma of the (k+1)-th selection indicating symbol is determined to vary according to an image z-coordinate of the (k+1)-th subject spatial relative coordinate point that represents a z-coordinate value corresponding to the (k+1)-th subject spatial relative coordinate point on the updated image z-axis.

As one example, each of the k-th visualized coordinate space and the (k+1)-th visualized coordinate space is a 3-dimensional space including a visualized x-axis, a visualized y-axis perpendicular to the visualized x-axis, and a visualized z-axis perpendicular to a plane formed by the visualized x-axis and the visualized y-axis, wherein each of the visualized x-axis, the visualized y-axis, and the visualized z-axis is set to correspond to the image x-axis, the image y-axis, and the image z-axis, and wherein (1) each of a (T_k)-th starting graphical element, the (T_k)-th current graphical element, and a (T_k)-th target graphical element is allowed to be displayed on the k-th visualized coordinate space, and (2) each of a (T_(k+1))-th starting graphical element, the (T_(k+1))-th current graphical element, and a (T_(k+1))-th target graphical element is allowed to be displayed on the (k+1)-th visualized coordinate space, wherein at least one of a color, a brightness, and a chroma of each of the (T_k)-th starting graphical element, the (T_k)-th current graphical element, the (T_k)-th target graphical element, the (T_(k+1))-th starting graphical element, the (T_(k+1))-th current graphical element, and the (T_(k+1))-th target graphical element is determined to vary according to each value of each visualized z-coordinate thereof that represents each coordinate value of a z-coordinate corresponding thereto on the visualized z-axis.

As one example, at the process of (I), the processor allows (i) k-th relative coordinates acquired by calculating coordinate values of the k-th subject spatial relative coordinate point while using the k-th subject spatial absolute coordinate point as its origin and (ii) k-th absolute coordinates acquired by converting coordinate values of the k-th relative coordinates within the subject space, to be displayed on at least part of the 1-st region of the display or on a 3-rd region of the display; and wherein at the process of (III), the processor allows (i) (k+1)-th relative coordinates acquired by calculating coordinate values of the (k+1)-th subject spatial relative coordinate point while using the (k+1)-th subject spatial absolute coordinate point as its origin and (ii) (k+1)-th absolute coordinates acquired by converting coordinate values of the (k+1)-th relative coordinates within the subject space, to be displayed on at least part of the 1-st region of the display or on the 3-rd region of the display by updating previous coordinates.

As one example, the external magnetic field is generated by a coil system connected to the user interface providing device, wherein the coil system includes a 1-st coil to a j-th coil, wherein each of the 1-st coil to the j-th coil is applied with each of a 1-st current to a j-th current, to thereby generate each of a 1-st sub-magnetic field to a j-th sub-magnetic field, wherein the external magnetic field is generated by overlapping each of the 1-st sub-magnetic field to the j-th sub-magnetic field; and wherein (i) each of the 1-st current to the j-th current to be applied to each of the 1-st coil the j-th coil at the (T_k_q)-th time point is determined by referring to the (T_k_q)-th yaw, the (T_k_p)-th pitch, and the (T_k_q)-th desired magnetic field strength corresponding to the instruction to generate the (T_k_q)-th magnetic field and (ii) each of the 1-st current to the j-th current to be applied to each of the 1-st coil the j-th coil at the (T_(k+1)_m)-th time point is determined by referring to the (T_(k+1)_m)-th yaw, the (T_(k+1)_m)-th pitch, and the (T_(k+1) m)-th desired magnetic field corresponding to the instruction to generate the (T_(k+1)_m)-th magnetic field.

As one example, on condition that the user interface providing device has stored each information on a measured magnetic flux density, which is measured in advance for each of specific coordinate points that are a plurality of points among all coordinate points of the subject space, in each of magnetic flux density tables corresponding to the specific coordinate points when a certain magnetic field as the external magnetic field is generated by applying a certain current as each of the 1-st current to the j-th current to each of the 1-st coil to the j-th coil, each of the magnetic flux density tables corresponding to a point P is represented as an array A(P), wherein the point P is one of the specific coordinate points and is the k-th subject spatial relative coordinate point or the (k+1)-th subject spatial relative coordinate point, wherein

$$A ( P ) = [ \begin{matrix}{A}_{x 1} ( P ) & {A}_{x 2} ( P ) & … & {A}_{xj} ( P ) \\ {A}_{y 1} ( P ) & {A}_{y 2} ( P ) & … & {A}_{y j} ( P ) \\ {A}_{z 1} ( P ) & {A}_{z 2} ( P ) & … & {A}_{z j} ( P )\end{matrix} ] ,$$

wherein the array A(P) includes Aₓ₁(P) to Aₓⱼ(P), A_{y1}(P) to A_{yj}(P), and A_{z1}(P) to A_{zj}(P), wherein Aₓⱼ(P) represents a (j_1)-st magnetic flux density of the j-th sub-magnetic field generated by the j-th coil in a first direction as an subject space x-axis with respect to the point P, A_{yj}(P) represents a (j_2)-nd magnetic flux density of the j-th sub-magnetic field generated by the j-th coil in a second direction as an subject space y-axis with respect to the point P, and A_{zj}(P) represents a (j_3)-rd magnetic flux density of the j-th sub-magnetic field generated by the j-th coil in a third direction as a direction perpendicular to a plane formed by the subject space x-axis and the subject space y-axis with respect to the point P; and wherein B_{ref}(P) represents the instruction to generate the (T_k_q)-th magnetic field or the instruction to generate the (T_(k+1)_m)-th magnetic field with respect to the point P, wherein the B_{ref}(P) and A(P)I* follow a relationship as below:

$${B}_{ref} ( P ) = [ \begin{matrix}{A}_{x 1} ( P ) & {A}_{x 2} ( P ) & … & {A}_{xj} ( P ) \\ {A}_{y 1} ( P ) & {A}_{y 2} ( P ) & … & {A}_{y j} ( P ) \\ {A}_{z 1} ( P ) & {A}_{z 2} ( P ) & … & {A}_{z j} ( P )\end{matrix} ] [ \begin{matrix}{i}_{1}^{*} \\ ⋮ \\ {i}_{j}^{*}\end{matrix} ] = A ( P ) {I}^{*}$$

wherein, I* is an array whose components include values of each of the 1-st current to the j-th current that is applied to each of the 1-st coil to the j-th coil, wherein each of ij to it represents values of each of the 1-st current to the j-th current, and wherein the 1-st current to the j-th current follow a formula below:

$${I}^{*} = [ \begin{matrix}{i}_{1}^{*} \\ ⋮ \\ {i}_{j}^{*}\end{matrix} ] = {A ( A {A}^{T} )}^{- 1} {B}_{r e f} ( P ) .$$

As one example, on condition that the k-th subject spatial relative coordinate point or the (k+1)-th subject spatial relative coordinate point is not one of the specific coordinate points, the processor performs a predetermined calibration, which includes a linear interpolation calibration, (i) on a magnetic flux density table corresponding to the k-th subject spatial relative coordinate point by referring to each of k-th specific magnetic flux density tables corresponding to at least two or more k-th specific coordinate points, which are within a predetermined distance from the k-th subject spatial relative coordinate point or (ii) on a magnetic flux density table corresponding to the (k+1)-th subject spatial relative coordinate point by referring to each of (k+1)-th specific magnetic flux density tables corresponding to at least two or more (k+1)-th specific coordinate points, which are within a predetermined distance from the (k+1)-th subject spatial relative coordinate point.chromachromachroma

In addition, recordable media that are readable by a computer for storing a computer program to execute the method of the present disclosure is further provided.

### BRIEF DESCRIPTION OF THE DRAWINGS

The following drawings to be used for explaining example embodiments of the present disclosure are only part of example embodiments of the present disclosure and other drawings can be acquired based on the drawings by those skilled in the art of the present disclosure without inventive work.

FIG. 1 is a drawing schematically illustrating a user interface providing device that provides a user interface capable of allowing a magnetic catheter to be controlled by using an external magnetic field in accordance with one example embodiment of the present disclosure.

FIG. 2A and FIG. 2B are drawings schematically illustrating a whole system that is allowed to be controlled by the user interface including a coil system, in accordance with one example embodiment of the present disclosure.

FIG. 3 is a flowchart schematically illustrating a flow of the user interface providing device capable of performing processes of acquiring a subject spatial image of a subject space that is affected by the external magnetic field, checking a position of the magnetic catheter, generating the external magnetic field, and then changing the external magnetic in accordance with one example embodiment of the present disclosure.

FIG. 4A and FIG. 4B are drawings schematically illustrating a coordinate space and coordinate axes to be used for changing a direction of the magnetic catheter within the subject space in a method of photographing the subject space affected by the external magnetic field in accordance with one example embodiment of the present disclosure.

FIG. 5A and FIG. 5B are drawings schematically illustrating the user interface capable of allowing the magnetic catheter to be controlled by using the external magnetic field, wherein the user interface is provided on a display by the user interface providing device in accordance with one example embodiment of the present disclosure.

### DETAILED DESCRIPTION OF THE PREFERRED EMBODIMENTS

In the following detailed description, reference is made to the accompanying drawings that show, by way of illustration, specific embodiments in which the invention may be practiced. These embodiments are described in sufficient detail to enable those skilled in the art to practice the invention. It is to be understood that the various embodiments of the present invention, although different, are not necessarily mutually exclusive. For example, a particular feature, structure, or characteristic described herein in connection with one embodiment may be implemented within other embodiments without departing from the spirit and scope of the present invention.

In addition, it is to be understood that the position or arrangement of individual elements within each disclosed embodiment may be modified without departing from the spirit and scope of the present invention. The following detailed description is, therefore, not to be taken in a limiting sense, and the scope of the present invention is defined only by the appended claims, appropriately interpreted, along with the full range of equivalents to which the claims are entitled. In the drawings, like numerals refer to the same or similar functionality throughout the several views.

To allow those skilled in the art to carry out the present invention easily, the example embodiments of the present invention by referring to attached diagrams will be explained in detail as shown below.

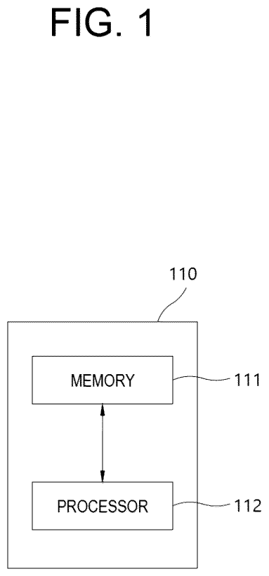

FIG. 1 is a drawing schematically illustrating a user interface providing device that provides a user interface capable of allowing a magnetic catheter to be controlled by using an external magnetic field in accordance with one example embodiment of the present disclosure.

Referring to FIG. 1, the user interface providing device 110 may include a memory 111 to store instructions to transmit and receive information such as a position of the magnetic catheter, a subject image, and a current required to generate the external magnetic field, and a processor 112 for controlling the magnetic catheter by generating the external magnetic field according to the instructions stored in the memory 110.

Specifically, the user interface providing device 110 may typically achieve a desired system performance by using combinations of at least one computing device and at least one computer software, e.g., a computer processor, a memory, a storage, an input device, an output device, or any other conventional computing components, an electronic communication device such as a router or a switch, an electronic information storage system such as a network-attached storage (NAS) device and a storage area network (SAN) as the computing device and any instructions that allow the computing device to function in a specific way as the computer software.

Also, the processors of such devices may include hardware configuration of MPU (Micro Processing Unit) or CPU (Central Processing Unit), cache memory, data bus, etc. Additionally, the computing device may further include operating system (OS) and software configuration of applications that achieve specific purposes.

Such description of the computing device does not exclude an integrated device including any combination of a processor, a memory, a medium, or any other computing components for implementing the present disclosure.

The configuration of the user interface providing device 110 for providing the user interface capable of allowing a user, e.g., a practitioner, to control the magnetic catheter by using the external magnetic field is described as above.

Below, a method of providing the user interface for allowing the user to control the magnetic catheter by using the external magnetic field will be described.

For reference, the below description will refer to a 3-dimensional cartesian coordinate system, however, the present disclosure is not limited thereto and may use a spherical coordinate system, another coordinate system or a combination thereof. It is to be appreciated that when the combination of different coordinate systems is used, a process of converting between the different coordinate systems may be additionally performed.

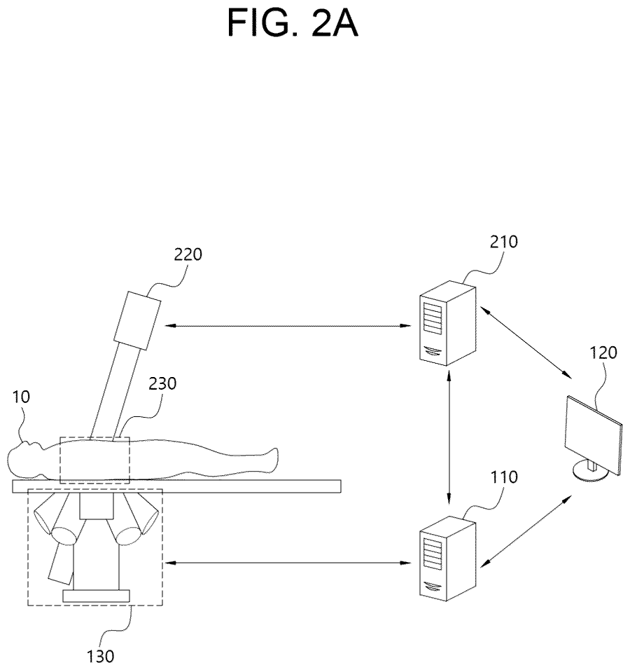

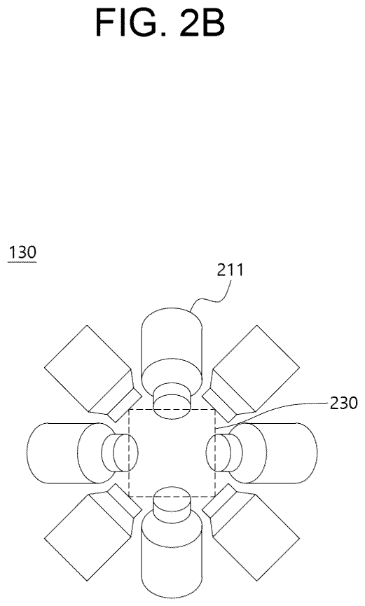

FIG. 2A and FIG. 2B are drawings schematically illustrating a whole system that is allowed to be controlled by the user interface, including a coil system, in accordance with one example embodiment of the present disclosure.

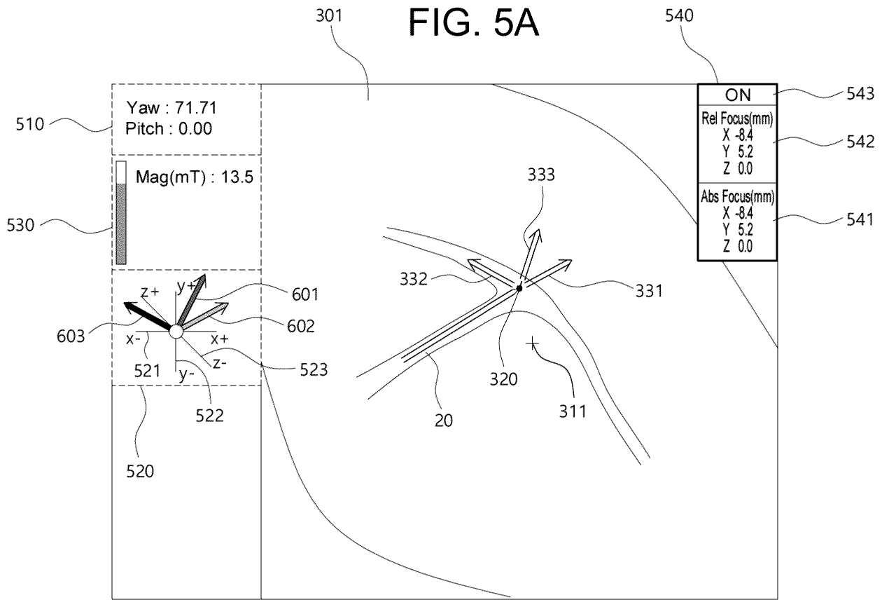

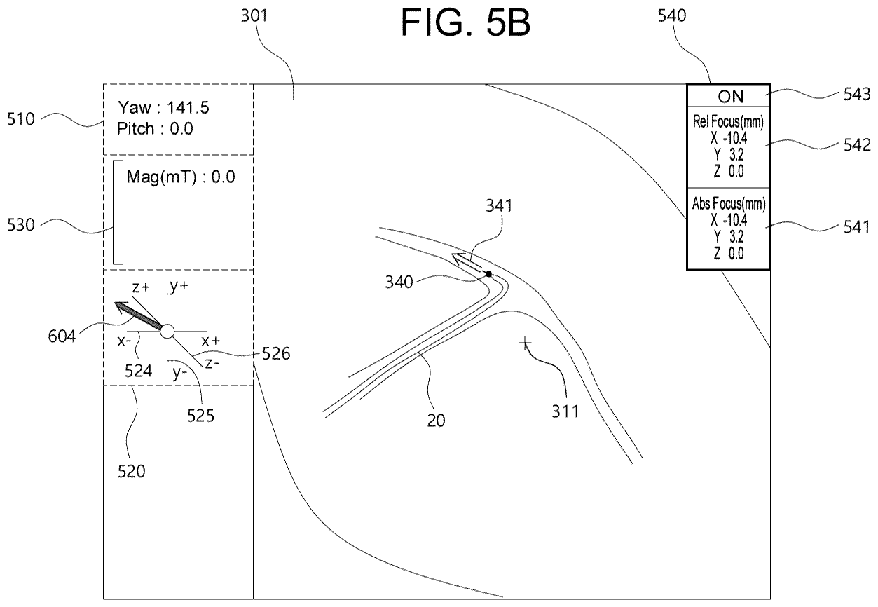

First, by referring to FIG. 2A, the user interface providing device 110 that provides the user interface capable of allowing the magnetic catheter to be controlled by using the external magnetic field may be connected to a display 120 for displaying the user interface. An example embodiment of the user interface is illustrated in FIG. 5A and FIG. 5B, which will be explained in detail below.

Further, the user interface providing device 110 may be connected to the coil system 130 to allow the external magnetic field to be generated or changed in a subject space 230 that includes a surgical region of a patient 10 receiving a surgical operation performed with the magnetic catheter. Herein, at least part of an end of the magnetic catheter of the present disclosure may be made with at least one material that bends when affected with a magnetic field, wherein the material may include at least part of N52, polydimthylsiloxane (PDMS), or a ferromagnetic material. Furthermore, by referring to a top view of the coil system 130 illustrated in FIG. 2B, the coil system 130 may include a plurality of coils 211 and each of the coils 211 may generate each of sub-magnetic fields that may overlap with each other within the subject space 230, to thereby generate or change the external magnetic field.

Moreover, the user interface providing device 110 may be connected to a subject spatial image managing device 210 to allow the user interface providing device 110 to obtain a subject spatial image from the subject spatial image managing device 210 and allow the user interface providing device 110 to provide the subject spatial image through the user interface. Herein, the subject spatial image is generated by photographing a part of the subject space 230 according to at least one predetermined photographing condition. Further, the subject spatial image managing device 210 may be connected to a subject spatial image photographing device 220 for photographing the subject space 230. Herein, the subject spatial image may be a 2-dimensional X-ray image, but it is not limited thereto.

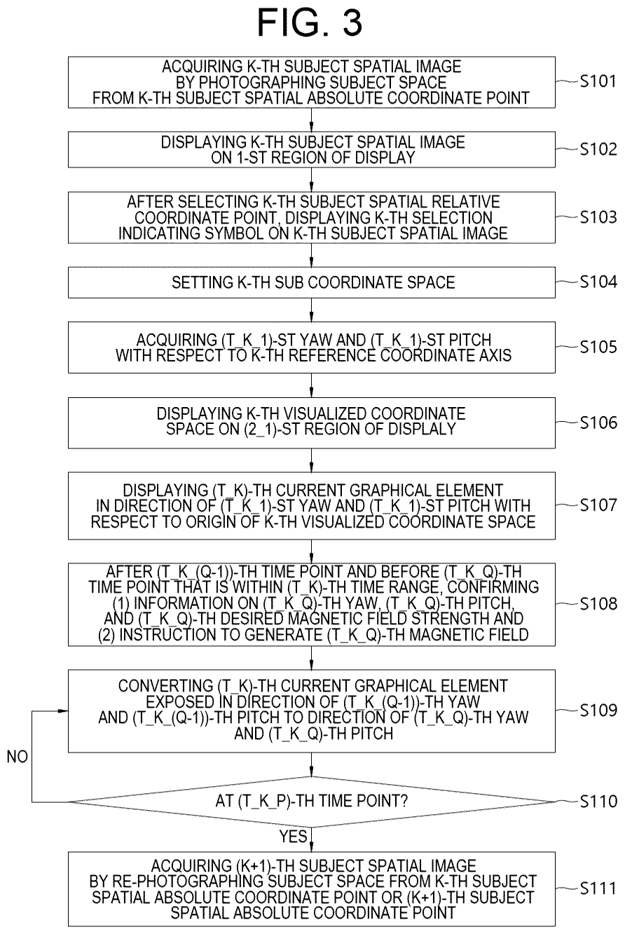

FIG. 3 is a flowchart schematically illustrating a flow of the user interface providing device 110 capable of performing processes of acquiring the subject spatial image of the subject space 230 that is affected by the external magnetic field, checking a position of the magnetic catheter, generating the external magnetic field, and then changing the external magnetic in accordance with one example embodiment of the present disclosure.

By referring to FIG. 3, the user interface providing device 110 may obtain a k-th subject spatial image from the subject spatial image managing device 210, at a step of S101. Herein, the k-th subject spatial image may be photographed from a k-th subject spatial absolute coordinate point of the subject space 230. Herein, the k-th subject spatial absolute coordinate point may correspond to a center point of the k-th subject spatial image.

Further, the user interface providing device 110 may allow the k-th subject spatial image to be displayed on a 1-st region of the display 120 of the user interface providing device 110, at a step of S102. FIG. 5A schematically illustrates the user interface being displayed on the display 120. It is noted that the k-th subject spatial image 301 is displayed on a 1-st region of the display 120 in accordance with one example embodiment of the present disclosure. Furthermore, FIG. 5A illustrates a center symbol corresponding to the k-th subject spatial absolute coordinate point 311, which serves as a reference point from which the k-th subject spatial image 301 is photographed, is also displayed along with the k-th subject spatial image 301 on the 1-st region of the display 120.

Next, the user interface providing device 110 may display a k-th selection indicating symbol at a position corresponding to a k-th subject spatial relative coordinate point as information on the position of the magnetic catheter. Herein, the k-th subject spatial relative coordinate point may be acquired by referring to the k-th subject spatial image 301 through a manual input of the user or an automatic method, at a step of S103.

Herein, the k-th subject spatial relative coordinate point is a point within the k-th subject space 230, and therefore, may be represented by using k-th absolute coordinates. However, in order to allow the user to easily recognize the position of the magnetic catheter, the user interface providing device 110 may calculate k-th relative coordinates, i.e., values as to how far it is from the k-th subject spatial absolute coordinate point 311 where the k-th subject spatial image 301 is photographed, to thereby display the k-th absolute coordinates 541 and the k-th relative coordinates 542 on the display 120. By referring to FIG. 5A again, the k-th absolute coordinates 541 and the k-th relative coordinates 542 are displayed on a specific region 540 in the 1-st region. It is to be noted that in FIG. 5A the k-th subject spatial absolute coordinate point 311 is used as the origin of the subject space 230, and accordingly the k-th absolute coordinates 541 and the k-th relative coordinates 542 are displayed in the same way. Further, the k-th absolute coordinates 541 and the k-th relative coordinates 542 are not limited to be displayed on the specific region 540 but may be displayed on a 3-rd region of the display 120.

Next, as another example, the user interface providing device 110 may display a system on/off state indication 543. The system on/off state indication 543 may allow the user to recognize whether the magnetic catheter can be controlled.

Further, as still another example, the user interface providing device 110 may display the k-th selection indicating symbol at the position corresponding to the k-th subject spatial relative coordinate point 320. Herein, the k-th selection indicating symbol may be displayed with a color, a brightness, and a chroma. Furthermore, the user interface providing device 110 may set a horizontal direction of the k-th subject spatial image 301 displayed on the display 120 as an image x-axis, a vertical direction of the k-th subject spatial image 301 displayed on the display 120 as an image y-axis, and a direction that is perpendicular to a plane formed by the image x-axis and the image y-axis as an image z-axis. Therefore, the user interface providing device 110 may vary at least a part of the color, the brightness, and the chroma of the k-th selection indicating symbol by referring to the k-th relative coordinates 542 of the k-th subject spatial relative coordinate point 320. For example, if the k-th relative coordinates 542 of the k-th subject spatial relative coordinate point 320 are represented as [image x-coordinate, image y-coordinate, image z-coordinate], at least part of a color, a brightness, and a chroma for coordinates (0,0,2) may be set to be have stroner/weaker color, higher/lower brightness, and higher/lower chroma than those for coordinates (0,0,1). Therefore, the user interface providing device 110 may allow the user to select the image z-coordinate even if the k-th subject spatial image 301 is a 2-dimensional image.

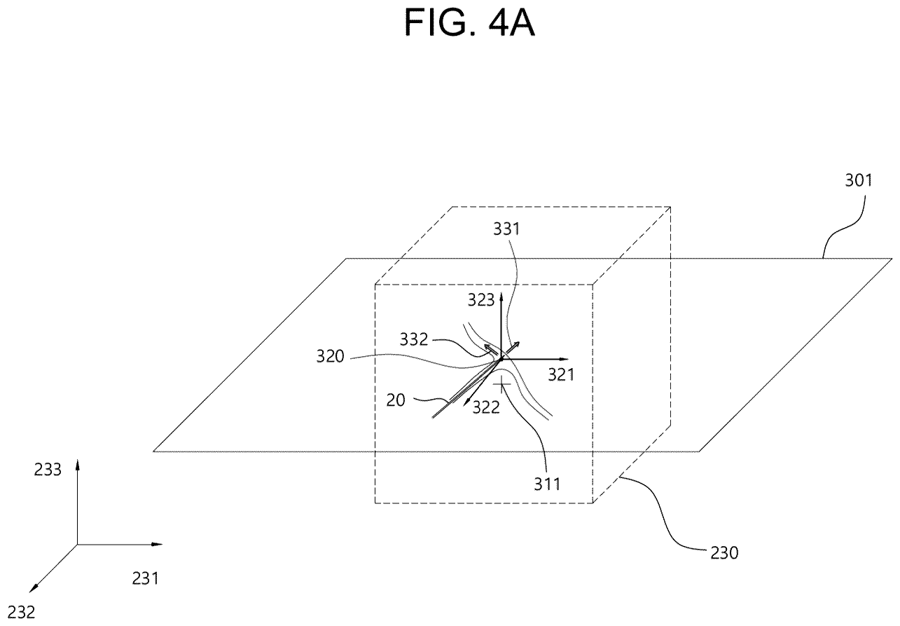

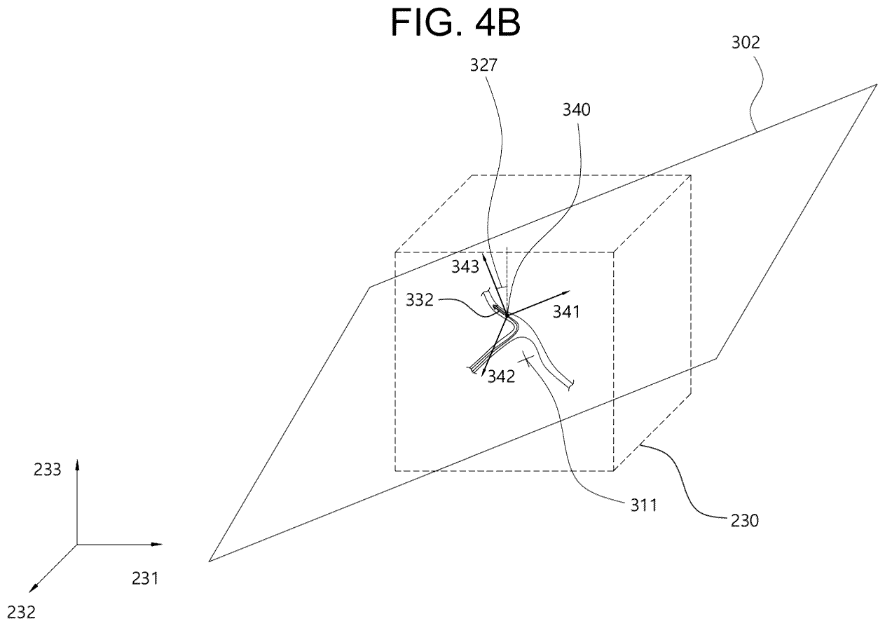

Next, the user interface providing device 110 may set a k-th sub coordinate space, at a step of S104. Herein, axes of the k-th sub coordinate space may be set by referring to coordinates of the k-th subject spatial image 301 and coordinates of the k-th subject spatial relative coordinate point 320. FIG. 4A schematically illustrates an example of how the subject space 230 that is affected by the external magnetic field is photographed and further illustrates directions, a coordinate space, and coordinate axes that need to be considered to change the direction of the magnetic catheter 20 in the subject space 230. By referring to FIG. 4A, the k-th sub coordinate space may have (1) a horizontal axis (x-axis) 321 defining coordinates in a horizontal direction of the k-th subject spatial image 301, (2) a vertical axis (y-axis) 322 defining coordinates in a vertical direction of the k-th subject spatial image 301, and (3) a perpendicular axis (z-axis) 323 defining coordinates in a direction perpendicular to a plane of the k-th subject spatial image 301, each of which considers the k-th subject spatial relative coordinate point 320 as its origin. For reference, a term “the sub coordinate space” is used to represent another coordinate space that is different from the subject space 230 defined by a subject space x-axis 231, a subject space y-axis 232, and a subject space z-axis 233 corresponding to the subject space 230. However, the space defined by the sub coordinate space is the same as the space defined by the subject space 230. Further, the user interface providing device 110 may set one of the coordinate axes (i.e. the horizontal axis 321, the vertical axis 322, and the perpendicular axis 323) of the k-th sub coordinate space to be a k-th reference coordinate axis depending on different embodiments of the present disclosure. Below, an example embodiment of setting an axis in a positive direction from an origin of the horizontal axis 321 as the k-th reference coordinate axis will be explained.

First, it is to be appreciated that a (T_k_1)-st time point represents a time point when the k-th subject spatial relative coordinate point 320 is selected, and each of a (T_k_2)-nd time point to a (T_k_p)-th time point represents each time point when the external magnetic field is applied to the k-th subject spatial relative coordinate point 320 or a change of the external magnetic field is made to the k-th subject spatial relative coordinate point 320. Herein, the (T_k_1)-st time point to the (T_k_p)-th time point are included in a (T_k)-th time range that starts from a time point of acquiring the k-th subject spatial image 301 to a time point of acquiring a (k+1)-th subject spatial image by re-photographing a part of the subject space 230 after a change in the position of the magnetic catheter 20 is made, with k being an integer greater than or equal to 1 and p being an integer greater than or equal to 2.

Next, the user interface providing device 110 may acquire information on a (T_k_1)-st yaw and a (T_k_1)-st pitch representing direction information of where the magnetic catheter is facing at the (T_k_1)-st time point with respect to the k-th reference coordinate axis, at a step of S105. Herein, the k-th reference coordinate axis is defined by an axis in a direction of having positive coordinate values on the horizontal-axis 321 with respect to the origin. Further, (i) the (T_k_1)-st yaw may represent a degree of rotation of the magnetic catheter 20 from the plane formed by the horizontal-axis 321 and the vertical-axis 322 with respect to the k-th reference coordinate axis 321 and (2) the (T_k_1)-st pitch may represent a degree of rotation of the magnetic catheter 20 from the perpendicular axis 323 with respect to the k-th reference coordinate axis. By referring back to FIG. 4A, the (T_k_1)-st yaw and the (T_k_1)-st pitch may be set to correspond to an initial direction of the magnetic catheter 20. It is to be appreciated that the (T_k_1)-st yaw and the (T_k_1)-st pitch may be determined by the user manually or measured by the user interface providing device 110 automatically according to various embodiments of the present disclosure.

Further, the user interface providing device 110 may display a k-th visualized coordinate space, which is acquired by visualizing the k-th sub coordinate space, on a (2_1)-st region of the display, at a step of S106. Herein, the k-th visualized coordinate space is a 3-dimensional space with a visualized x-axis, a visualized y-axis perpendicular to the visualized x-axis, and a visualized z-axis perpendicular to a plane formed by the visualized x-axis and the visualized y-axis. Herein, each of the visualized x-axis, the visualized y-axis, and the visualized z-axis may be set to correspond to the image x-axis, the image y-axis, and the image z-axis in order to represent the k-th visualized coordinate space to be corresponding to the k-th subject spatial image 301. By referring back to FIG. 5A, it illustrates the k-th visualized coordinate space including the visualized x-axis 521, the visualized y-axis 522, and the visualized z-axis 523 on the (2_1)-st region 520 that is located at a middle of a left side of the display 120, and it is to be noted that the visualized x-axis 521, the visualized y-axis 522, and the visualized z-axis 523 respectively correspond to the image x-axis, the image y-axis, and the image z-axis of the k-th subject spatial image 301 that is displayed on a right side of the display 120.

Furthermore, the user interface providing device 110 may expose a (T_k)-th current graphical element in a direction corresponding to the (T_k_1)-st yaw and the (T_k_1)-st pitch with respect to an origin of the k-th visualized coordinate space, at a step of S107, in order to allow the user to recognize the direction of the magnetic catheter 20 at the (T_k_1)-st time point. Then, the (T_k)-th current graphical element may be generated and changed according to the direction of the external magnetic field at each of the (T_k_2)-nd time point to the (T_k_p)-th time point included in the (T_k)-th time range. This will be explained later.

Moreover, the user interface providing device 110 may acquire, during a time section of after a (T_k_(q−1))-th time point and before a (T_k_q)-th time point, (1) information on a (T_k_q)-th magnetic field to be generated at the (T_k_q)-th time point with respect to the k-th subject spatial relative coordinate point 320, and (2) an instruction to generate the (T_k_q)-th magnetic field, wherein the information on the (T_k_q)-th magnetic field includes a (T_k_q)-th yaw, a (T_k_q)-th pitch, and a (T_k_q)-th desired magnetic field strength of the (T_k_q)-th magnetic field, at a step of S108. Herein the (T_k_q)-th time point is a specific time point within a time range starting from the (T_k_2)-nd time point to the (T_k_p)-th time point, with q being an integer greater than or equal to 2 and less than or equal to p. Further, each of the (T_k_2)-nd time point to the (T_k_p)-th time point may be set to be equally apart from each other, but they are not limited thereto. As an example, a gap between the (T_k_(q−1))-th time point and the (T_k_q)-th time point may be set depending on (i) the information on the (T_k_q)-th magnetic field including the (T_k_q)-th yaw, the (T_k_q)-th pitch, and the (T_k_q)-th desired magnetic field strength of the (T_k_q)-th magnetic field and (ii) whether the instruction to generate the (T_k_q)-th magnetic field is acquired.

Next, in response to acquiring (i) the information on the (T_k_q)-th magnetic field to be generated, which includes the (T_k_q)-th yaw, the (T_k_q)-th pitch, and the (T_k_q)-th desired magnetic field strength of the (T_k_q)-th magnetic field, and (ii) the instruction to generate the (T_k_q)-th magnetic field, at the (T_k_q)-th time point, the user interface providing device 110 may change the (T_k)-th current graphical element, which has been exposed in a direction of a (T_k_(q−1))-th yaw and a (T_k_(q−1))-th pitch corresponding to the (T_k_(q−1))-th time point, to be exposed in a direction of the (T_k_q)-th yaw and the (T_k_q)-th pitch corresponding to the (T_k_q)-th time point, by referring to the (T_k_q)-th yaw, the (T_k_q)-th pitch, and the (T_k_q)-th desired magnetic field strength, at a step of S109. Herein, the user interface providing device 110 may update each of the (T_k_q)-th pitch to the (T_k_p)-th pitch and the (T_k_q)-th desired magnetic field strength, to thereby display each of the (T_k_q)-th pitch to the (T_k_p)-th pitch on a (2_2)-nd region of the display 120 and display the (T_k_q)-th desired magnetic field strength on a (2_3)-rd region of the display 120. That is, as time passes from the (T_k_2)-nd time point to the (T_k_p)-th time point, the direction of the (T_k)-th current graphical element displayed on the (2_1)-st region of the display 120 is continuously updated and displayed thereon according to the direction of the external magnetic field at each of the (T_k_2)-nd time point to the (T_k_p)-th time point. Therefore, the user interface providing device 110 may check whether the (T_k_p)-th time point is reached, at a step of S110, and continuously update and display the (T_k)-th current graphical element at each of the (T_k_2)-nd time point to the (T_k_p)-th time point.

By referring back to FIG. 5A, it illustrates one example of the user interface when the external magnetic field with a specific direction 333 is applied to the k-th subject spatial relative coordinate point 320 after a certain time has passed from the (T_k_1)-st time point. It is to be noted that the (T_k)-th current graphical element 601 is exposed in the k-th visualized coordinate space on the (2_1)-st region 520 of the display 120 such that it corresponds to the specific direction 333. And then, the direction of the (T_k)-th current graphical element 601 may be continuously updated as time passes.

As one example embodiment of the present disclosure, if a target direction of the magnetic catheter 20 at the (T_k_p)-th time point with respect to the k-th subject spatial relative coordinate point 320 is determined in advance, wherein the (T_k_p)-th time point is a last time point included in the (T_k)-th time range, a direction of a (T_k_p)-th magnetic field (to be generated at the (T_k_p)-th time point to correspond to the target direction) with respect to the k-th subject spatial relative coordinate point 320 may also be determined, therefore, the user interface providing device 110 may acquire, in advance, information on a (T_k_p)-th yaw and a (T_k_p)-th pitch corresponding to the direction of the (T_k_p)-th magnetic field. By referring back to FIG. 4A, if the magnetic catheter 20 is determined to face a left direction 332 from a branching point at the (T_k_p)-th time point, the user interface providing device 110 may acquire the information on the (T_k_p)-th yaw and the (T_k_p)-th pitch corresponding to the left direction 332 of the branching point. Herein, information on the (T_k_p)-th yaw and the (T_k_p)-th pitch corresponding to the target direction may be manually inputted by the user or may be automatically determined by the user interface providing device 110, but the present disclosure is not limited thereto. Further, the user interface providing device 110 may expose a (T_k)-th starting graphical element in the direction corresponding to the (T_k_1)-st yaw and the (T_k_1)-st pitch, and expose a (T_k)-th target graphical element in a direction corresponding to the (T_k_p)-th yaw and the (T_k_p)-th pitch, to thereby allow the user to easily recognize a current direction of the magnetic catheter 20 and its corresponding current direction of the external magnetic field while displaying the current direction of the external magnetic field to be compared with the starting direction of the external magnetic field and the target direction of the external magnetic field. By referring back to FIG. 5A, the (T_k)-th starting graphical element 602 is exposed in the starting direction 331 corresponding to the (T_k_1)-st yaw and the (T_k_1)-st pitch in the k-th visualized coordinate space on the (2_1)-st region 520 of the display 120 and the (T_k)-th target graphical element 603 is also exposed in the target direction 332 corresponding to the (T_k_p)-th yaw and the (T_k_p)-th pitch acquired beforehand by the user interface providing device 110.

As another example, at least one of a color, a brightness, and a chroma of each of the (T_k)-th starting graphical element 602, the (T_k)-th current graphical element 601, and the (T_k)-th target graphical element 603 is determined to vary according to each value of each visualized z-coordinate thereof that represents each coordinate value of a z-coordinate corresponding thereto on the visualized z-axis 523 of the k-th visualized coordinate space. For example, the (T_k)-th starting graphical element 602, the (T_k)-th current graphical element 601, and the (T_k)-th target graphical element 603 may be represented as different colors, and they may be set to have different brightness or chroma depending on how much each of values of each visualized z-coordinate 523 thereof is tilted towards a positive direction or a negative direction.

Next, at the (T_k_p)-th time point, the (T_k_p)-th magnetic field may be generated for the k-th subject spatial relative coordinate point 320, and the user interface providing device 110 may re-photograph a part of the subject space 230 at a new location where the magnetic catheter 20 is arrived after the (T_k_p)-th time point, to thereby acquire the (k+1)-th subject spatial image 302 from the subject spatial image managing device 210, at a step of S111. Herein the (k+1)-th subject spatial image may be re-photographed from the k-th subject spatial absolute coordinate point 311 or may be re-photographed from a (k+1)-th subject spatial absolute coordinate point that is different from the k-th subject spatial absolute coordinate point 311. Therefore, a (k+1)-th subject spatial relative coordinate point corresponding to a changed position, i.e., the new location, of the magnetic catheter 20 may be divided into two cases. That is, (i) in case the (k+1)-th subject spatial image is re-photographed from the k-th subject spatial absolute coordinate point 311, the (k+1)-th subject spatial relative coordinate point is a relative coordinate point with respect to the k-th subject spatial absolute coordinate point and (ii) in case the (k+1)-th subject spatial image is re-photographed from the (k+1)-th subject spatial absolute coordinate point that is different from the k-th subject spatial absolute coordinate point 311, the (k+1)-th subject spatial relative coordinate point is a relative coordinate point with respect to the (k+1)-th subject spatial absolute coordinate point.

FIG. 4B illustrates the (k+1)-th subject spatial image 302 acquired with a change in a photographing condition of the k-th subject spatial image 301. According to FIG. 4B, the (k+1)-th subject spatial image 302 may be photographed from the k-th subject spatial absolute coordinate point 311 (from which the k-th subject spatial image 301 was also photographed), but with a changed shooting angle 327. In case the (k+1)-th subject spatial relative coordinate point 340 is selected as the changed position, i.e., the new location, of the magnetic catheter 20, new coordinate axes 341, 342, and 343 of a (k+1)-th sub coordinate space with the (k+1)-th subject spatial relative coordinate point 340 being considered as its origin are tilted from the coordinate axes 321, 322, and 323 of the k-th sub coordinate space by the changed shooting angle 327. Further, the (k+1)-th subject spatial relative coordinate point 340 may be the relative coordinate point with respect to the k-th subject spatial absolute coordinate point 311.

Furthermore, by referring to FIG. 5B, it can be seen that (1) the (k+1)-th subject spatial image 302 re-photographed as mentioned above is displayed on the 1-st region of the display 102, (2) a (k+1)-th visualized coordinate with an updated visualized x-axis 524, an updated visualized y-axis 525, and an updated visualized z-axis 526 corresponding to the coordinate axes 341, 342, and 343 of the (k+1)-th sub coordinate space are displayed on the (2_1)-st region 520. Further, a (T_(k+1)_1)-st yaw and a (T_(k+1)_1)-st pitch with respect to a (k+1)-th reference coordinate axis in a positive direction of a horizontal axis 341 among the coordinate axes 341, 342, and 343 of the (k+1)-th sub coordinate space are displayed on the (2_2)-nd region 510 of the display 120.

Furthermore, since the instruction to generate the external magnetic field is not yet received at a (T_(k+1)_1)-st time point, a desired magnetic field strength is displayed as 0 on the (2_3)-rd region 530 of the display 120. Moreover, a (T_(k+1))-th current graphical element 604 is exposed in the direction corresponding to the (T_(k+1)_1)-st yaw and the (T_(k+1)_1)-st pitch with respect to the (k+1)-th reference coordinate axis. Herein, since the (k+1)-th subject spatial image 302 is acquired with the changed shooting angle 327 compared to the k-th subject spatial image 301, at least one of a color, a brightness, and a chroma of the (T_(k+1))-th current graphical element can be set to be different from the (T_k)-th current graphical element.

Moreover, since the (T_k_p)-th magnetic field generated for the k-th subject spatial relative coordinate point 320 at the (T_k_p)-th time point affects its surroundings within a predetermined magnetic range, in case the magnetic catheter 20 moves within the predetermined magnetic range from the k-th subject spatial relative coordinate point 320 after the (T_k_p)-th time point, the magnetic catheter 20 may move with the direction thereof being maintained. And if the movement of the magnetic catheter 20 is completed, the user interface providing device 110 may allow a coil system 130 to stop generating the (T_k_p)-th magnetic field and then obtain the (k+1)-th subject spatial image 302 from the subject spatial image managing device 210. That is, after the (T_k_p)-th time point, in case the magnetic catheter 20 having been located at the k-th subject spatial relative coordinate point 320 moves and arrives at the (k+1)-th subject spatial relative coordinate point 340 which is identically affected by the direction of the (T_k_p)-th magnetic field having been generated for the k-th subject spatial relative coordinate point 320, the user interface providing device 110 may set the (T_(k+1)_1)-st yaw and the (T_(k+1)_1)-st pitch to be in the same direction as the direction of the magnetic catheter 20 at the (T_(k+1)_1)-st time point by referring to the (T_k_p)-th yaw, the (T_k_p)-th pitch and the k-th reference coordinate axis. Herein, the (T_(k+1)_1)-st time point is a time point of the (k+1)-th subject spatial relative coordinate point 340 being selected as a new location of the magnetic catheter 20. For example, referring back to FIG. 4B, in case the (k+1)-th subject spatial image 302 is re-photographed with the changed shooting angle 327 compared to the k-th subject spatial image 301, since (i) the (T_k_p)-th yaw and the (T_k_p)-th pitch is represented with respect to the k-th reference coordinate axis of the k-th sub coordinate space and (ii) the (k+1)-th reference coordinate axis of the (k+1)-th sub coordinate space corresponding to the (k+1)-th subject spatial image 302 is different from the k-th reference coordinate axis by the changed shooting angle 327, the user interface providing device 110 may set the (T_(k+1)_p)-th yaw and the (T_(k+1)_p)-th pitch by referring to the (T_k_p)-th yaw, the (T_k_p)-th pitch, and the k-th reference coordinate axis.

Processes to be performed after setting the (k+1)-th sub coordinate space in response to the selection of the (k+1)-th subject spatial relative coordinate point 340 are similar to processes performed after setting the k-th sub coordinate space, therefore details thereof are omitted.

Below, processes of generating the external magnetic field by the coil system 130 that is connected to the user interface providing device 110 in accordance with one example embodiment of the present disclosure are explained.

For reference, since the processes of generating and changing the external magnetic field by the coil system 130 during the (T_k)-th time range, which is a time range of from a time of acquiring the k-th subject spatial image 301 to a time of acquiring the (k+1)-th subject spatial image 302, may be similarly applied to the (T_(k+1))-th time range, which is a time range of from a time of acquiring the (k+1)-th subject spatial image 302 to a time of acquiring the (k+2)-th subject spatial image, detailed explanations on the processes during (T_(k+1))-th time range will be omitted.

The external magnetic field may be generated and changed by the coil system 130 including a 1-st coil to a j-th coil 211 that is connected to the user interface providing device 110. Herein, each of the 1-st coil to the j-th coil is applied with each of a 1-st current to a j-th current, to thereby generate each of a 1-st sub-magnetic field to a j-th sub-magnetic field. Therefore, the external magnetic field may be generated by overlapping each of the 1-st sub-magnetic field to the j-th sub-magnetic field. The 1-st current to the j-th current to be applied to the 1-st coil to the j-th coil may be determined by referring to the (T_k_q)-th yaw, the (T_k_q)-th pitch, and the (T_k_q)-th desired magnetic field strength corresponding to the instruction to generate the (T_k_q)-th magnetic field.

Herein, according to one example embodiment of the present disclosure, the user interface providing device 110 may have stored each information on a measured magnetic flux density, which is measured in advance for each of specific coordinate points that are a plurality of points among all coordinate points of the subject space 230, in each of magnetic flux density tables corresponding to the specific coordinate points when a certain magnetic field as the external magnetic field is generated by applying a certain current as each of the 1-st current to the j-th current to each of the 1-st coil to the j-th coil. Further, for a point P as the k-th subject spatial relative coordinate point 320, which is one of the specific coordinate points, one of the magnetic flux density tables corresponding to the point P may be represented as an array A(P) as below.

$$A ( P ) = [ \begin{matrix}{A}_{x 1} ( P ) & {A}_{x 2} ( P ) & … & {A}_{xj} ( P ) \\ {A}_{y 1} ( P ) & {A}_{y 2} ( P ) & … & {A}_{y j} ( P ) \\ {A}_{z 1} ( P ) & {A}_{z 2} ( P ) & … & {A}_{z j} ( P )\end{matrix} ] ,$$

Herein, the array A(P) includes Aₓ₁(P) to Aₓⱼ(P), A_{y1}(P) to A_{yj}(P), and A_{z1}(P) to A_{zj}(P), wherein Aₓⱼ(P) represents a (j_1)-st magnetic flux density of the j-th sub-magnetic field generated by the j-th coil 211 in a first direction as the subject space x-axis 231 with respect to the point P, A_{yj}(P) represents a (j_2)-nd magnetic flux density of the j-th sub-magnetic field generated by the j-th coil 211 in a second direction as the subject space y-axis 232 with respect to the point P, and A_{zj}(P) represents a (j_3)-rd magnetic flux density of the j-th sub-magnetic field generated by the j-th coil 211 in a third direction as a direction perpendicular to a plane formed by the subject space x-axis 231 and the subject space y-axis 232 (i.e., the subject space z-axis 233) with respect to the point P.

Further, if B_{ref}(P) represents the instruction to generate the (T_k_q)-th magnetic field with respect to the point P, The B_{ref}(P) and A(P)I* may follow a relationship as below:

$${B}_{ref} ( P ) = [ \begin{matrix}{A}_{x 1} ( P ) & {A}_{x 2} ( P ) & … & {A}_{xj} ( P ) \\ {A}_{y 1} ( P ) & {A}_{y 2} ( P ) & … & {A}_{y j} ( P ) \\ {A}_{z 1} ( P ) & {A}_{z 2} ( P ) & … & {A}_{z j} ( P )\end{matrix} ] [ \begin{matrix}{i}_{1}^{*} \\ ⋮ \\ {i}_{j}^{*}\end{matrix} ] = A ( P ) {I}^{*}$$

Herein, I* is an array whose components include values of each of the 1-st current to the j-th current to be applied to each of the 1-st coil to the j-th coil, wherein each of ij to i; represents values of each of the 1-st current to the j-th current.

Therefore, the 1-st current to the j-th current may follow a formula below:

$${I}^{*} = [ \begin{matrix}{i}_{1}^{*} \\ ⋮ \\ {i}_{j}^{*}\end{matrix} ] = {A ( A {A}^{T} )}^{- 1} {B}_{r e f} ( P ) .$$

It is to be noted that the k-th subject spatial relative coordinate point may not be one of the specific coordinate points. In that case, the user interface providing device 110 may perform a predetermined calibration on a magnetic flux density table corresponding to the k-th subject spatial relative coordinate point 320 by referring to each of k-th specific magnetic flux density tables corresponding to at least two or more k-th specific coordinate points, which are within a predetermined distance from the k-th subject spatial relative coordinate point 320. Herein, the predetermined calibration may be a linear interpolation, but the present disclosure is not limited thereto.

According to the method of the present disclosure, there is no need to develop a new device for displaying and managing visualized information on individual subject spaces for surgical operations and the catheter in the subject spaces, thus existing X-ray imaging devices can be utilized.

The present disclosure has an effect of providing the user interface that can interact with the user to allow the user to check a position of the magnetic catheter and to control a change in the direction of the magnetic catheter by controlling the direction of the external magnetic field.

The present disclosure has another effect of providing the user interface that can continuously display information on the direction of the external magnetic field used for changing the direction of the magnetic catheter.

The present disclosure still has still another effect of providing the user interface capable of allowing a magnetic field generating device to accurately calculate each of required currents to be applied to each of coils in order to generate the external magnetic field used for changing the direction of the magnetic catheter.

The embodiments of the present disclosure as explained above can be implemented in a form of executable program command through a variety of computer means recordable in computer readable media. The computer readable media may include solely or in combination, program commands, data files, and data structures. The program commands recorded to the media may be components specially designed for the present disclosure or may be usable to a skilled human in a field of computer software. Computer readable media include magnetic media such as hard disk, floppy disk, and magnetic tape, optical media such as CD-ROM and DVD, magneto-optical media such as floptical disk and hardware devices such as ROM, RAM, and flash memory specially designed to store and carry out program commands. Program commands may include not only a machine language code made by a complier but also a high-level code that can be used by an interpreter etc., which is executed by a computer. The aforementioned hardware device can work as more than a software module to perform the action of the present disclosure and they can do the same in the opposite case.

As seen above, the present disclosure has been explained by specific matters such as detailed components, limited embodiments, and drawings. They have been provided only to help more general understanding of the present disclosure. It, however, will be understood by those skilled in the art that various changes and modification may be made from the description without departing from the spirit and scope of the disclosure as defined in the following claims.

Accordingly, the thought of the present disclosure must not be confined to the explained embodiments, and the following patent claims as well as everything including variations equal or equivalent to the patent claims pertain to the category of the thought of the present disclosure.

What is claimed is:

1. A method for providing a user interface, comprising steps of:
(a) in response to acquiring a k-th subject spatial image, which is obtained by photographing a part of subject space from a k-th subject spatial absolute coordinate point through a subject spatial image managing device connected to a user interface providing device, wherein k is an integer greater than or equal to 1, and wherein the subject space is affected by an external magnetic field, (i) displaying, by the user interface providing device, the k-th subject spatial image on a 1-st region of a display of the user interface providing device, and (ii) in response to acquiring selection information of a k-th subject spatial relative coordinate point, which is a relative coordinate point with respect to the k-th subject spatial absolute coordinate point, corresponding to a position of a magnetic catheter positioned within the subject space, displaying, by the user interface providing device, a k-th selection indicating symbol at a position corresponding to the k-th subject spatial relative coordinate point in the k-th subject spatial image; and
(b) on condition that a (T_k_1)-st time point represents a time point when the k-th subject spatial relative coordinate point is selected, and each of a (T_k_2)-nd time point to a (T_k_p)-th time point represents each time point when the external magnetic field is applied upon or a change of the external magnetic field is made to the k-th subject spatial relative coordinate point, wherein the (T_k_1)-st time point to the (T_k_p)-th time point are within a (T_k)-th time range that starts from a time point of acquiring the k-th subject spatial image to a time point of acquiring a (k+1)-th subject spatial image by re-photographing a part of the subject space after a change in a position of the magnetic catheter is made, with p being an integer greater than or equal to 2, (b1) (i) setting, by the user interface providing device, a k-th sub coordinate space having each of coordinate axes corresponding to the k-th subject spatial image while using the k-th subject spatial relative coordinate point as its origin by referring to the k-th subject spatial image and the k-th subject spatial relative coordinate point, and (ii) acquiring, by the user interface providing device, information on a (T_k_1)-st yaw and a (T_k_1)-st pitch representing direction information of where the magnetic catheter is facing at the (T_k_1)-st time point with respect to a k-th reference coordinate axis, which is a specific coordinate axis among coordinate axes corresponding to the k-th sub coordinate space, (b2) (i) displaying, by the user interface providing device, a k-th visualized coordinate space, which is acquired by visualizing the k-th sub coordinate space, on a (2_1)-st region of the display, and (ii) displaying, by the user interface providing device, a (T_k)-th current graphical element to be exposed in a direction corresponding to the (T_k_1)-st yaw and the (T_k_1)-st pitch with respect to an origin of the k-th visualized coordinate space; and (b3) in response to confirming, during a time section of after a (T_k_(q−1))-th time point and before a (T_k_q)-th time point, (1) information on a (T_k_q)-th magnetic field to be generated at the (T_k_q)-th time point with respect to the k-th subject spatial relative coordinate point, and (2) an instruction to generate the (T_k_q)-th magnetic field, wherein the information on the (T_k_q)-th magnetic field includes a (T_k_q)-th yaw, a (T_k_q)-th pitch, and a (T_k_q)-th desired magnetic field strength of the (T_k_q)-th magnetic field, and wherein the (T_k_q)-th time point is a specific time point within a time range starting from the (T_k_2)-nd time point to the (T_k_p)-th time point, with q being an integer greater than or equal to 2 and less than or equal to p, (i) at the (T_k_q)-th time point, changing, by the user interface providing device, the (T_k)-th current graphical element, which has been exposed in a direction of a (T_k_(q−1))-th yaw and a (T_k_(q−1))-th pitch corresponding to the (T_k_(q−1))-th time point, to be exposed in a direction of the (T_k_q)-th yaw and the (T_k_q)-th pitch corresponding to the (T_k_q)-th time point, by referring to the (T_k_q)-th yaw, the (T_k_q)-th pitch, and the (T_k_q)-th desired magnetic field strength, (ii) updating, by the user interface providing device, each of the (T_k_q)-th pitch to the (T_k_p)-th pitch, to thereby display thereof on a (2_2)-nd region of the display, and (iii) continuously displaying, by the user interface providing device, the (T_k_q)-th desired magnetic field strength on a (2_3)-rd region of the display starting from the (T_k_2)-nd time point to the (T_k_p)-th time point.

2. The method of claim 1, wherein, after the step of (b), the method further comprises steps of:
(c) after a generation of a (T_k_p)-th magnetic field at the (T_k_p)-th time point with respect to the k-th subject spatial relative coordinate point, in response to acquiring the (k+1)-th subject spatial image by re-photographing a part of the subject space due to a change in the position of the magnetic catheter through the subject spatial image managing device, (i) displaying, by the user interface providing device, the (k+1)-th subject spatial image on the 1-st region of the display, and (ii) in response to acquiring selection information of a (k+1)-th subject spatial relative coordinate point corresponding to a changed position of the magnetic catheter, displaying, by the user interface providing device, a (k+1)-th selection indicating symbol at a position corresponding to the (k+1)-th subject spatial relative coordinate point on the (k+1)-th subject spatial image, wherein, in case the (k+1)-th subject spatial image is re-photographed from the k-th subject spatial absolute coordinate point, the (k+1)-th subject spatial relative coordinate point is a relative coordinate point with respect to the k-th subject spatial absolute coordinate point and wherein, in case the (k+1)-th subject spatial image is re-photographed from a (k+1)-th subject spatial absolute coordinate point that is different from the k-th subject spatial absolute coordinate point, the (k+1)-th subject spatial relative coordinate point is a relative coordinate point with respect to the (k+1)-th subject spatial absolute coordinate point; and
(d) on condition that a (T_(k+1)_1)-st time point represents a time point when the (k+1)-th subject spatial relative coordinate point is selected, and each of a (T_(k+1)_2)-nd time point to a (T_(k+1)_m)-th time point represents each time point when the external magnetic field is applied upon or a change of the external magnetic field is made to the (k+1)-th subject spatial relative point, wherein the (T_(k+1)_1)-st time point to the (T_(k+1)_m)-th time point is within a (T_(k+1))-th time range that starts from a time point of acquiring the (k+1)-th subject spatial image to a time point of acquiring a (k+2)-th subject spatial image by re-photographing a part of the subject space after a change in a position of the magnetic catheter is made, with m being an integer greater than or equal to 2, (d1) (i) setting, by the user interface providing device, a (k+1)-th sub coordinate space having each of coordinate axes corresponding to the (k+1)-th subject spatial image while using the (k+1)-th subject spatial relative coordinate point as its origin by referring to the (k+1)-th subject spatial image and the (k+1)-th subject spatial relative coordinate point, and (ii) acquiring, by the user interface providing device, information on a (T_(k+1)_1)-st yaw and a (T_(k+1)_1)-st pitch representing direction information of where the magnetic catheter is facing at the (T_(k+1)_1)-st time point with respect to a (k+1)-th reference coordinate axis, which is a specific coordinate axis among coordinate axes corresponding to the (k+1)-th sub coordinate space, (d2) (i) displaying, by the user interface providing device, a (k+1)-th visualized coordinate space, which is acquired by visualizing the (k+1)-th sub coordinate space, on the (2_1)-st region of the display, and (ii) displaying, by the user interface providing device, a (T_(k+1))-th current graphical element to be exposed in a direction corresponding to the (T_(k+1)_1)-st yaw and the (T_(k+1)_1)-st pitch with respect to an origin of the (k+1)-th visualized coordinate space; and (d3) in response to confirming, during a time section of after a (T_(k+1)_(n−1))-th time point and before a (T_(k+1)_n)-th time point, (1) information on a (T_(k+1)_n)-th magnetic field to be generated at the (T_(k+1)_n)-th time point with respect to the (k+1)-th subject spatial relative coordinate point, and (2) an instruction to generate the (T_(k+1)_n)-th magnetic field, wherein the information on the (T_(k+1)_n)-th magnetic field includes a (T_(k+1)_n)-th yaw, a (T_(k+1)_n)-th pitch, and a (T_(k+1)_n)-th desired magnetic field strength of the (T_(k+1)_n)-th magnetic field, and wherein the (T_(k+1)_n)-th time point is a specific time point within a time range starting from the (T_(k+1)_2)-nd time point to the (T_(k+1) m)-th time point, with n being an integer greater than or equal to 2 and less than or equal to m, (i) at the (T_(k+1)_n)-th time point, changing, by the user interface providing device, the (T_(k+1))-th current graphical element, which has been exposed in a direction of a (T_(k+1)_(n−1))-th yaw and a (T_(k+1)_(n−1))-th pitch corresponding to the (T_(k+1)_(n−1))-th time point, to be exposed in a direction of the (T_(k+1)_n)-th yaw and the (T_(k+1)_n)-th pitch corresponding to the (T_(k+1)_n)-th time point, by referring to the (T_(k+1)_n)-th yaw, the (T_(k+1)_n)-th pitch, and the (T_(k+1)_n)-th desired magnetic field strength, (ii) updating, by the user interface providing device, each of the (T_(k+1)_n)-th pitch to the (T_(k+1)_m)-th pitch, to thereby display thereof on the (2_2)-nd region of the display, and (iii) continuously displaying, by the user interface providing device, the (T_(k+1)_n)-th desired magnetic field strength on the (2_3)-rd region of the display starting from the (T_(k+1)_2)-nd time point to the (T_(k+1)_m)-th time point.

3. The method of claim 2, wherein, at the step of (b1), the user interface providing device acquires target direction information, including a (T_k_p)-th yaw and a (T_k_p)-th pitch, corresponding to the (T_k_p)-th magnetic field to be generated at the (T_k_p)-th time point on the k-th subject spatial relative coordinate point, to thereby allow, at the step of (b2), (i) a (T_k)-th starting graphical element to be exposed in a direction corresponding to the (T_k_1)-st yaw and the (T_k_1)-st pitch with respect to the origin of the k-th visualized coordinate space, and (ii) a (T_k)-th target graphical element to be exposed in a direction corresponding to the (T_k_p)-th yaw and the (T_k_p)-th pitch with respect to the origin of the k-th visualized coordinate space; and wherein, at the step of (d1), the user interface providing device acquires target direction information, including a (T_(k+1)_m)-th yaw and a (T_(k+1)_m)-th pitch, corresponding to a (T_(k+1)_m)-th magnetic field to be generated at the (T_(k+1) m)-th time point on the (k+1)-th subject spatial relative coordinate point, to thereby allow, at the step of (d2), (i) a (T_(k+1))-th starting graphical element to be exposed in a direction corresponding to a (T_(k+1)_1)-st yaw and a (T_(k+1)_1)-st pitch with respect to the origin of the (k+1)-th visualized coordinate space, and (ii) a (T_(k+1))-th target graphical element to be exposed in a direction corresponding to the (T_(k+1)_m)-th yaw and the (T_(k+1)_m)-th pitch with respect to the origin of the (k+1)-th visualized coordinate space.

4. The method of claim 3, wherein, at the step of (d1), the user interface providing device sets the (T_(k+1)_1)-st yaw and the (T_(k+1)_1)-st pitch representing direction information of where the magnetic catheter is facing at the (T_(k+1)_1)-st time point with respect to the (k+1)-th reference coordinate axis to be same as a direction corresponding to the (T_k_p)-th yaw and the (T_k_p)-th pitch with respect to the k-th reference coordinate axis, by referring to (1) the (T_k_p)-th yaw and the (T_k_p)-th pitch corresponding to the (T_k_p)-th magnetic field generated at the (T_k_p)-th time point on the k-th subject spatial relative coordinate point and (2) the k-th reference coordinate axis.

5. The method of claim 2, wherein, at the step of (a), the user interface providing device (1) sets a (k_1)-st direction corresponding to a horizontal direction of the k-th subject spatial image as an image x-axis, a (k_2)-nd direction that is perpendicular to the (k_1)-st direction of the k-th subject spatial image as an image y-axis, and a direction that is perpendicular to a plane formed by the image x-axis and the image y-axis as an image z-axis, and (2) displays the k-th selection indicating symbol corresponding to the k-th subject spatial relative coordinate point on the k-th subject spatial image, wherein at least a part of a color, a brightness, and a chroma of the k-th selection indicating symbol is determined to vary according to a predetermined condition depending on an image z-coordinate of the k-th subject spatial relative coordinate point that represents a z-coordinate value corresponding to the k-th subject spatial relative coordinate point on the image z-axis; and wherein, at the step of (c), the user interface providing device (3) updates a ((k+1)_1)-st direction corresponding to a horizontal direction of the (k+1)-th subject spatial image to be the image x-axis, updates a ((k+1)_2)-nd direction corresponding to a vertical direction of the (k+1)-th subject spatial image that is perpendicular to the ((k+1)−1)-st direction to be the image y-axis, and updates a direction that is perpendicular to the plane formed by the updated image x-axis and the updated image y-axis to be the image z-axis, and (4) displays the (k+1)-th selection indicating symbol corresponding to the (k+1)-th subject spatial relative coordinate point on the (k+1)-th subject spatial image, wherein at least a part of color, a brightness, and a chroma of the (k+1)-th selection indicating symbol is determined to vary according to an image z-coordinate of the (k+1)-th subject spatial relative coordinate point that represents a z-coordinate value corresponding to the (k+1)-th subject spatial relative coordinate point on the updated image z-axis.

6. The method of claim 5, wherein each of the k-th visualized coordinate space and the (k+1)-th visualized coordinate space is a 3-dimensional space including a visualized x-axis, a visualized y-axis perpendicular to the visualized x-axis, and a visualized z-axis perpendicular to a plane formed by the visualized x-axis and the visualized y-axis, wherein each of the visualized x-axis, the visualized y-axis, and the visualized z-axis is set to correspond to the image x-axis, the image y-axis, and the image z-axis, and wherein (1) each of a (T_k)-th starting graphical element, the (T_k)-th current graphical element, and a (T_k)-th target graphical element is allowed to be displayed on the k-th visualized coordinate space, and (2) each of a (T_(k+1))-th starting graphical element, the (T_(k+1))-th current graphical element, and a (T_(k+1))-th target graphical element is allowed to be displayed on the (k+1)-th visualized coordinate space, wherein at least one of a color, a brightness, and a chroma of each of the (T_k)-th starting graphical element, the (T_k)-th current graphical element, the (T_k)-th target graphical element, the (T_(k+1))-th starting graphical element, the (T_(k+1))-th current graphical element, and the (T_(k+1))-th target graphical element is determined to vary according to each value of each visualized z-coordinate thereof that represents each coordinate value of a z-coordinate corresponding thereto on the visualized z-axis.

7. The method of claim 2, wherein, at the step of (a), the user interface providing device allows (i) k-th relative coordinates acquired by calculating coordinate values of the k-th subject spatial relative coordinate point while using the k-th subject spatial absolute coordinate point as its origin and (ii) k-th absolute coordinates acquired by converting coordinate values of the k-th relative coordinates within the subject space, to be displayed on at least part of the 1-st region of the display or on a 3-rd region of the display; and wherein at the step of (c), the user interface providing device allows (i) (k+1)-th relative coordinates acquired by calculating coordinate values of the (k+1)-th subject spatial relative coordinate point while using the (k+1)-th subject spatial absolute coordinate point as its origin and (ii) (k+1)-th absolute coordinates acquired by converting coordinate values of the (k+1)-th relative coordinates within the subject space, to be displayed on at least part of the 1-st region of the display or on the 3-rd region of the display by updating previous coordinates.

8. The method of claim 2, wherein the external magnetic field is generated by a coil system connected to the user interface providing device, wherein the coil system includes a 1-st coil to a j-th coil, wherein each of the 1-st coil to the j-th coil is applied with each of a 1-st current to a j-th current, to thereby generate each of a 1-st sub-magnetic field to a j-th sub-magnetic field, wherein the external magnetic field is generated by overlapping each of the 1-st sub-magnetic field to the j-th sub-magnetic field; and wherein (i) each of the 1-st current to the j-th current to be applied to each of the 1-st coil the j-th coil at the (T_k_q)-th time point is determined by referring to the (T_k_q)-th yaw, the (T_k_p)-th pitch, and the (T_k_q)-th desired magnetic field strength corresponding to the instruction to generate the (T_k_q)-th magnetic field and (ii) each of the 1-st current to the j-th current to be applied to each of the 1-st coil the j-th coil at the (T_(k+1) m)-th time point is determined by referring to the (T_(k+1)_m)-th yaw, the (T_(k+1) m)-th pitch, and the (T_(k+1) m)-th desired magnetic field corresponding to the instruction to generate the (T_(k+1)_m)-th magnetic field.

9. The method of claim 8, wherein, on condition that the user interface providing device has stored each information on a measured magnetic flux density, which is measured in advance for each of specific coordinate points that are a plurality of points among all coordinate points of the subject space, in each of magnetic flux density tables corresponding to the specific coordinate points when a certain magnetic field as the external magnetic field is generated by applying a certain current as each of the 1-st current to the j-th current to each of the 1-st coil to the j-th coil, each of the magnetic flux density tables corresponding to a point P is represented as an array A(P), wherein the point P is one of the specific coordinate points and is the k-th subject spatial relative coordinate point or the (k+1)-th subject spatial relative coordinate point,
wherein
$$A ( P ) = [ \begin{matrix}{A}_{x 1} ( P ) & {A}_{x 2} ( P ) & … & {A}_{xj} ( P ) \\ {A}_{y 1} ( P ) & {A}_{y 2} ( P ) & … & {A}_{y j} ( P ) \\ {A}_{z 1} ( P ) & {A}_{z 2} ( P ) & … & {A}_{z j} ( P )\end{matrix} ] ,$$
wherein the array A(P) includes Aₓ₁(P) to Aₓⱼ(P), A_{y1}(P) to A_{yj}(P), and A_{z1}(P) to A_{zj}(P), wherein Aₓⱼ(P) represents a (j_1)-st magnetic flux density of the j-th sub-magnetic field generated by the j-th coil in a first direction as an subject space x-axis with respect to the point P, A_{yj}(P) represents a (j_2)-nd magnetic flux density of the j-th sub-magnetic field generated by the j-th coil in a second direction as an subject space y-axis with respect to the point P, and A_{zj}(P) represents a (j_3)-rd magnetic flux density of the j-th sub-magnetic field generated by the j-th coil in a third direction as a direction perpendicular to a plane formed by the subject space x-axis and the subject space y-axis with respect to the point P; and
wherein B_{ref}(P) represents the instruction to generate the (T_k_q)-th magnetic field or the instruction to generate the (T_(k+1)_m)-th magnetic field with respect to the point P, wherein the B_{ref}(P) and A(P)I* follow a relationship as below:
$${B}_{ref} ( P ) = [ \begin{matrix}{A}_{x 1} ( P ) & {A}_{x 2} ( P ) & … & {A}_{xj} ( P ) \\ {A}_{y 1} ( P ) & {A}_{y 2} ( P ) & … & {A}_{y j} ( P ) \\ {A}_{z 1} ( P ) & {A}_{z 2} ( P ) & … & {A}_{z j} ( P )\end{matrix} ] [ \begin{matrix}{i}_{1}^{*} \\ ⋮ \\ {i}_{j}^{*}\end{matrix} ] = A ( P ) {I}^{*}$$
wherein, I* is an array whose components include values of each of the 1-st current to the j-th current that is applied to each of the 1-st coil to the j-th coil, wherein each of ij to if represents values of each of the 1-st current to the j-th current, and wherein the 1-st current to the j-th current follow a formula below:
$${I}^{*} = [ \begin{matrix}{i}_{1}^{*} \\ ⋮ \\ {i}_{j}^{*}\end{matrix} ] = {A ( A {A}^{T} )}^{- 1} {B}_{r e f} ( P ) .$$

10. The method of claim 9, wherein, on condition that the k-th subject spatial relative coordinate point or the (k+1)-th subject spatial relative coordinate point is not one of the specific coordinate points, the user interface providing device performs a predetermined calibration, which includes a linear interpolation calibration, (i) on a magnetic flux density table corresponding to the k-th subject spatial relative coordinate point by referring to each of k-th specific magnetic flux density tables corresponding to at least two or more k-th specific coordinate points, which are within a predetermined distance from the k-th subject spatial relative coordinate point or (ii) on a magnetic flux density table corresponding to the (k+1)-th subject spatial relative coordinate point by referring to each of (k+1)-th specific magnetic flux density tables corresponding to at least two or more (k+1)-th specific coordinate points, which are within a predetermined distance from the (k+1)-th subject spatial relative coordinate point.

11. A user interface providing device for providing a user interface, comprising:
at least one memory that stores instructions;
at least one processor configured to execute the instructions to perform processes of: (I) in response to acquiring a k-th subject spatial image, which is obtained by photographing a part of subject space from a k-th subject spatial absolute coordinate point through a subject spatial image managing device connected to the user interface providing device, wherein k is an integer greater than or equal to 1, and wherein the subject space is affected by an external magnetic field, (i) displaying the k-th subject spatial image on a 1-st region of a display of the user interface providing device, and (ii) in response to acquiring selection information of a k-th subject spatial relative coordinate point, which is a relative coordinate point with respect to the k-th subject spatial absolute coordinate point, corresponding to a position of a magnetic catheter positioned within the subject space, displaying a k-th selection indicating symbol at a position corresponding to the k-th subject spatial relative coordinate point in the k-th subject spatial image; and (II) on condition that a (T_k_1)-st time point represents a time point when the k-th subject spatial relative coordinate point is selected, and each of a (T_k_2)-nd time point to a (T_k_p)-th time point represents each time point when the external magnetic field is applied upon or a change of the external magnetic field is made to the k-th subject spatial relative coordinate point, wherein the (T_k_1)-st time point to the (T_k_p)-th time point are within a (T_k)-th time range that starts from a time point of acquiring the k-th subject spatial image to a time point of acquiring a (k+1)-th subject spatial image by re-photographing a part of the subject space after a change in a position of the magnetic catheter is made, with p being an integer greater than or equal to 2, (II-1) (i) setting a k-th sub coordinate space having each of coordinate axes corresponding to the k-th subject spatial image while using the k-th subject spatial relative coordinate point as its origin by referring to the k-th subject spatial image and the k-th subject spatial relative coordinate point, and (ii) acquiring information on a (T_k_1)-st yaw and a (T_k_1)-st pitch representing direction information of where the magnetic catheter is facing at the (T_k_1)-st time point with respect to a k-th reference coordinate axis, which is a specific coordinate axis among coordinate axes corresponding to the k-th sub coordinate space, (II-2) (i) displaying a k-th visualized coordinate space, which is acquired by visualizing the k-th sub coordinate space, on a (2_1)-st region of the display, and (ii) displaying a (T_k)-th current graphical element to be exposed in a direction corresponding to the (T_k_1)-st yaw and the (T_k_1)-st pitch with respect to an origin of the k-th visualized coordinate space; and (II-3) in response to confirming, during a time section of after a (T_k_(q−1))-th time point and before a (T_k_q)-th time point, (1) information on a (T_k_q)-th magnetic field to be generated at the (T_k_q)-th time point with respect to the k-th subject spatial relative coordinate point, and (2) an instruction to generate the (T_k_q)-th magnetic field, wherein the information on the (T_k_q)-th magnetic field includes a (T_k_q)-th yaw, a (T_k_q)-th pitch, and a (T_k_q)-th desired magnetic field strength of the (T_k_q)-th magnetic field, and wherein the (T_k_q)-th time point is a specific time point within a time range starting from the (T_k_2)-nd time point to the (T_k_p)-th time point, with q being an integer greater than or equal to 2 and less than or equal to p, (i) at the (T_k_q)-th time point, changing the (T_k)-th current graphical element, which has been exposed in a direction of a (T_k_(q−1))-th yaw and a (T_k_(q−1))-th pitch corresponding to the (T_k_(q−1))-th time point, to be exposed in a direction of the (T_k_q)-th yaw and the (T_k_q)-th pitch corresponding to the (T_k_q)-th time point, by referring to the (T_k_q)-th yaw, the (T_k_q)-th pitch, and the (T_k_q)-th desired magnetic field strength, (ii) updating each of the (T_k_q)-th pitch to the (T_k_p)-th pitch, to thereby display thereof on a (2_2)-nd region of the display, and (iii) continuously displaying the (T_k_q)-th desired magnetic field strength on a (2_3)-rd region of the display starting from the (T_k_2)-nd time point to the (T_k_p)-th time point.

12. The user interface providing device of claim 11, wherein, after the process of (II), the processor further performs processes of:
(III) after a generation of a (T_k_p)-th magnetic field at the (T_k_p)-th time point with respect to the k-th subject spatial relative coordinate point, in response to acquiring the (k+1)-th subject spatial image by re-photographing a part of the subject space due to a change in the position of the magnetic catheter through the subject spatial image managing device, (i) displaying the (k+1)-th subject spatial image on the 1-st region of the display, and (ii) in response to acquiring selection information of a (k+1)-th subject spatial relative coordinate point corresponding to a changed position of the magnetic catheter, displaying a (k+1)-th selection indicating symbol at a position corresponding to the (k+1)-th subject spatial relative coordinate point on the (k+1)-th subject spatial image, wherein, in case the (k+1)-th subject spatial image is re-photographed from the k-th subject spatial absolute coordinate point, the (k+1)-th subject spatial relative coordinate point is a relative coordinate point with respect to the k-th subject spatial absolute coordinate point and wherein, in case the (k+1)-th subject spatial image is re-photographed from a (k+1)-th subject spatial absolute coordinate point that is different from the k-th subject spatial absolute coordinate point, the (k+1)-th subject spatial relative coordinate point is a relative coordinate point with respect to the (k+1)-th subject spatial absolute coordinate point; and (IV) on condition that a (T_(k+1)_1)-st time point represents a time point when the (k+1)-th subject spatial relative coordinate point is selected, and each of a (T_(k+1)_2)-nd time point to a (T_(k+1) m)-th time point represents each time point when the external magnetic field is applied upon or a change of the external magnetic field is made to the (k+1)-th subject spatial relative coordinate point, wherein the (T_(k+1)_1)-st time point to the (T_(k+1)_m)-th time point is within a (T_(k+1))-th time range that starts from a time point of acquiring the (k+1)-th subject spatial image to a time point of acquiring a (k+2)-th subject spatial image by re-photographing a part of the subject space after a change in a position of the magnetic catheter is made, with m being an integer greater than or equal to 2, (IV-1) (i) setting a (k+1)-th sub coordinate space having each of coordinate axes corresponding to the (k+1)-th subject spatial image while using the (k+1)-th subject spatial relative coordinate point as its origin by referring to the (k+1)-th subject spatial image and the (k+1)-th subject spatial relative coordinate point, and (ii) acquiring information on a (T_(k+1)_1)-st yaw and a (T_(k+1)_1)-st pitch representing direction information of where the magnetic catheter is facing at the (T_(k+1)_1)-st time point with respect to a (k+1)-th reference coordinate axis, which is a specific coordinate axis among coordinate axes corresponding to the (k+1)-th sub coordinate space, (IV-2) (i) displaying a (k+1)-th visualized coordinate space, which is acquired by visualizing the (k+1)-th sub coordinate space, on the (2_1)-st region of the display, and (ii) displaying a (T_(k+1))-th current graphical element to be exposed in a direction corresponding to the (T_(k+1)_1)-st yaw and the (T_(k+1)_1)-st pitch with respect to an origin of the (k+1)-th visualized coordinate space; and (IV-3) in response to confirming, during a time section of after a (T_(k+1)_(n−1))-th time point and before a (T_(k+1)_n)-th time point, (1) information on a (T_(k+1)_n)-th magnetic field to be generated at the (T_(k+1)_n)-th time point with respect to the (k+1)-th subject spatial relative coordinate point, and (2) an instruction to generate the (T_(k+1)_n)-th magnetic field, wherein the information on the (T_(k+1)_n)-th magnetic field includes a (T_(k+1)_n)-th yaw, a (T_(k+1)_n)-th pitch, and a (T_(k+1)_n)-th desired field magnetic strength of the (T_(k+1)_n)-th magnetic field, and wherein the (T_(k+1)_n)-th time point is a specific time point within a time range starting from the (T_(k+1)_2)-nd time point to the (T_(k+1)_m)-th time point, with n being an integer greater than or equal to 2 and less than or equal to m, (i) at the (T_(k+1)_n)-th time point, changing the (T_(k+1))-th current graphical element, which has been exposed in a direction of a (T_(k+1)_(n−1))-th yaw and a (T_(k+1)_(n−1))-th pitch corresponding to the (T_(k+1)_(n−1))-th time point, to be exposed in a direction of the (T_(k+1)_n)-th yaw and the (T_(k+1)_n)-th pitch corresponding to the (T_(k+1)_n)-th time point, by referring to the (T_(k+1)_n)-th yaw, the (T_(k+1)_n)-th pitch, and the (T_(k+1)_n)-th desired magnetic field strength, (ii) updating each of the (T_(k+1)_n)-th pitch to the (T_(k+1)_m)-th pitch, to thereby display thereof on the (2_2)-nd region of the display, and (iii) continuously displaying the (T_(k+1)_n)-th desired magnetic field strength on the (2_3)-rd region of the display starting from the (T_(k+1)_2)-nd time point to the (T_(k+1)_m)-th time point.

13. The user interface providing device of claim 12, wherein, at the process of (II-1), the processor acquires target direction information, including a (T_k_p)-th yaw and a (T_k_p)-th pitch, corresponding to the (T_k_p)-th magnetic field to be generated at the (T_k_p)-th time point on the k-th subject spatial relative coordinate point, to thereby allow, at the process of (II-2), (i) a (T_k)-th starting graphical element to be exposed in a direction corresponding to the (T_k_1)-st yaw and the (T_k_1)-st pitch with respect to the origin of the k-th visualized coordinate space, and (ii) a (T_k)-th target graphical element to be exposed in a direction corresponding to the (T_k_p)-th yaw and the (T_k_p)-th pitch with respect to the origin of the k-th visualized coordinate space; and wherein, at the process of (IV-1), the processor acquires target direction information, including a (T_(k+1)_m)-th yaw and a (T_(k+1)_m)-th pitch, corresponding to a (T_(k+1)_m)-th magnetic field to be generated at the (T_(k+1)_m)-th time point on the (k+1)-th subject spatial relative coordinate point, to thereby allow, at the process of (IV-2), (i) a (T_(k+1))-th starting graphical element to be exposed in a direction corresponding to a (T_(k+1)_1)-st yaw and a (T_(k+1)_1)-st pitch with respect to the origin of the (k+1)-th visualized coordinate space, and (ii) a (T_(k+1))-th target graphical element to be exposed in a direction corresponding to the (T_(k+1)_m)-th yaw and the (T_(k+1)_m)-th pitch with respect to the origin of the (k+1)-th visualized coordinate space.

14. The user interface providing device of claim 13, wherein, at the process of (IV-1), the processor sets the (T_(k+1)_1)-st yaw and the (T_(k+1)_1)-st pitch representing direction information of where the magnetic catheter is facing at the (T_(k+1)_1)-st time point with respect to the (k+1)-th reference coordinate axis to be same as a direction corresponding to the (T_k_p)-th yaw and the (T_k_p)-th pitch with respect to the k-th reference coordinate axis, by referring to (1) the (T_k_p)-th yaw and the (T_k_p)-th pitch corresponding to the (T_k_p)-th magnetic field generated at the (T_k_p)-th time point on the k-th subject spatial relative coordinate point and (2) the k-th reference coordinate axis.

15. The user interface providing device of claim 12, wherein, at the process of (I), the processor (1) sets a (k_1)-st direction corresponding to a horizontal direction of the k-th subject spatial image as an image x-axis, a (k_2)-nd direction that is perpendicular to the (k_1)-st direction of the k-th subject spatial image as an image y-axis, and a direction that is perpendicular to a plane formed by the image x-axis and the image y-axis as an image z-axis, and (2) displays the k-th selection indicating symbol corresponding to the k-th subject spatial relative coordinate point on the k-th subject spatial image, wherein at least a part of a color, a brightness, and a chroma of the k-th selection indicating symbol is determined to vary according to a predetermined condition depending on an image z-coordinate of the k-th subject spatial relative coordinate point that represents a z-coordinate value corresponding to the k-th subject spatial relative coordinate point on the image z-axis; and wherein, at the process of (III), the processor (3) updates a ((k+1)_1)-st direction corresponding to a horizontal direction of the (k+1)-th subject spatial image to be the image x-axis, updates a ((k+1)_2)-nd direction corresponding to a vertical direction of the (k+1)-th subject spatial image that is perpendicular to the ((k+1)−1)-st direction to be the image y-axis, and updates a direction that is perpendicular to the plane formed by the updated image x-axis and the updated image y-axis to be the image z-axis, and (4) displays the (k+1)-th selection indicating symbol corresponding to the (k+1)-th subject spatial relative coordinate point on the (k+1)-th subject spatial image, wherein at least a part of color, a brightness, and a chroma of the (k+1)-th selection indicating symbol is determined to vary according to an image z-coordinate of the (k+1)-th subject spatial relative coordinate point that represents a z-coordinate value corresponding to the (k+1)-th subject spatial relative coordinate point on the updated image z-axis.

16. The user interface providing device of claim 15, wherein each of the k-th visualized coordinate space and the (k+1)-th visualized coordinate space is a 3-dimensional space including a visualized x-axis, a visualized y-axis perpendicular to the visualized x-axis, and a visualized z-axis perpendicular to a plane formed by the visualized x-axis and the visualized y-axis, wherein each of the visualized x-axis, the visualized y-axis, and the visualized z-axis is set to correspond to the image x-axis, the image y-axis, and the image z-axis, and wherein (1) each of a (T_k)-th starting graphical element, the (T_k)-th current graphical element, and a (T_k)-th target graphical element is allowed to be displayed on the k-th visualized coordinate space, and (2) each of a (T_(k+1))-th starting graphical element, the (T_(k+1))-th current graphical element, and a (T_(k+1))-th target graphical element is allowed to be displayed on the (k+1)-th visualized coordinate space, wherein at least one of a color, a brightness, and a chroma of each of the (T_k)-th starting graphical element, the (T_k)-th current graphical element, the (T_k)-th target graphical element, the (T_(k+1))-th starting graphical element, the (T_(k+1))-th current graphical element, and the (T_(k+1))-th target graphical element is determined to vary according to each value of each visualized z-coordinate thereof that represents each coordinate value of a z-coordinate corresponding thereto on the visualized z-axis.

17. The user interface providing device of claim 12, wherein, at the process of (I), the processor allows (i) k-th relative coordinates acquired by calculating coordinate values of the k-th subject spatial relative coordinate point while using the k-th subject spatial absolute coordinate point as its origin and (ii) k-th absolute coordinates acquired by converting coordinate values of the k-th relative coordinates within the subject space, to be displayed on at least part of the 1-st region of the display or on a 3-rd region of the display; and wherein at the process of (III), the processor allows (i) (k+1)-th relative coordinates acquired by calculating coordinate values of the (k+1)-th subject spatial relative coordinate point while using the (k+1)-th subject spatial absolute coordinate point as its origin and (ii) (k+1)-th absolute coordinates acquired by converting coordinate values of the (k+1)-th relative coordinates within the subject space, to be displayed on at least part of the 1-st region of the display or on the 3-rd region of the display by updating previous coordinates.

18. The user interface providing device of claim 12, wherein the external magnetic field is generated by a coil system connected to the user interface providing device, wherein the coil system includes a 1-st coil to a j-th coil, wherein each of the 1-st coil to the j-th coil is applied with each of a 1-st current to a j-th current, to thereby generate each of a 1-st sub-magnetic field to a j-th sub-magnetic field, wherein the external magnetic field is generated by overlapping each of the 1-st sub-magnetic field to the j-th sub-magnetic field; and wherein (i) each of the 1-st current to the j-th current to be applied to each of the 1-st coil the j-th coil at the (T_k_q)-th time point is determined by referring to the (T_k_q)-th yaw, the (T_k_p)-th pitch, and the (T_k_q)-th desired magnetic field strength corresponding to the instruction to generate the (T_k_q)-th magnetic field and (ii) each of the 1-st current to the j-th current to be applied to each of the 1-st coil the j-th coil at the (T_(k+1) m)-th time point is determined by referring to the (T_(k+1)_m)-th yaw, the (T_(k+1)_m)-th pitch, and the (T_(k+1) m)-th desired magnetic field corresponding to the instruction to generate the (T_(k+1)_m)-th magnetic field.

19. The user interface providing device of claim 18, wherein, on condition that the user interface providing device has stored each information on a measured magnetic flux density, which is measured in advance for each of specific coordinate points that are a plurality of points among all coordinate points of the subject in each of magnetic flux density tables corresponding to the specific coordinate points when a certain magnetic field as the external magnetic field is generated by applying a certain current as each of the 1-st current to the j-th current to each of the 1-st coil to the j-th coil, each of the magnetic flux density tables corresponding to a point P is represented as an array A(P), wherein the point P is one of the specific coordinate points and is the k-th subject spatial relative coordinate point or the (k+1)-th subject spatial relative coordinate point,
wherein
$$A ( P ) = [ \begin{matrix}{A}_{x 1} ( P ) & {A}_{x 2} ( P ) & … & {A}_{xj} ( P ) \\ {A}_{y 1} ( P ) & {A}_{y 2} ( P ) & … & {A}_{y j} ( P ) \\ {A}_{z 1} ( P ) & {A}_{z 2} ( P ) & … & {A}_{z j} ( P )\end{matrix} ] ,$$
wherein the array A(P) includes Aₓ₁(P) to Aₓⱼ(P), A_{y1}(P) to A_{yj}(P), and A_{z1}(P) to A_{zj}(P), wherein Aₓⱼ(P) represents a (j_1)-st magnetic flux density of the j-th sub-magnetic field generated by the j-th coil in a first direction as an subject space x-axis with respect to the point P, A_{yj}(P) represents a (j_2)-nd magnetic flux density of the j-th sub-magnetic field generated by the j-th coil in a second direction as an subject space y-axis with respect to the point P, and A_{zj}(P) represents a (j_3)-rd magnetic flux density of the j-th sub-magnetic field generated by the j-th coil in a third direction as a direction perpendicular to a plane formed by the subject space x-axis and the subject space y-axis with respect to the point P; and
wherein B_{ref}(P) represents the instruction to generate the (T_k_q)-th magnetic field or the instruction to generate the (T_(k+1)_m)-th magnetic field with respect to the point P, wherein the B_{ref}(P) and A(P)I* follow a relationship as below:
$${B}_{ref} ( P ) = [ \begin{matrix}{A}_{x 1} ( P ) & {A}_{x 2} ( P ) & … & {A}_{xj} ( P ) \\ {A}_{y 1} ( P ) & {A}_{y 2} ( P ) & … & {A}_{y j} ( P ) \\ {A}_{z 1} ( P ) & {A}_{z 2} ( P ) & … & {A}_{z j} ( P )\end{matrix} ] [ \begin{matrix}{i}_{1}^{*} \\ ⋮ \\ {i}_{j}^{*}\end{matrix} ] = A ( P ) {I}^{*}$$
wherein, I* is an array whose components include values of each of the 1-st current to the j-th current that is applied to each of the 1-st coil to the j-th coil, wherein each of ij to ij represents values of each of the 1-st current to the j-th current, and wherein the 1-st current to the j-th current follow a formula below:
$${I}^{*} = [ \begin{matrix}{i}_{1}^{*} \\ ⋮ \\ {i}_{j}^{*}\end{matrix} ] = {A ( A {A}^{T} )}^{- 1} {B}_{r e f} ( P ) .$$

20. The user interface providing device of claim 19, wherein, on condition that the k-th subject spatial relative coordinate point or the (k+1)-th subject spatial relative coordinate point is not one of the specific coordinate points, the processor performs a predetermined calibration, which includes a linear interpolation calibration, (i) on a magnetic flux density table corresponding to the k-th subject spatial relative coordinate point by referring to each of k-th specific magnetic flux density tables corresponding to at least two or more k-th specific coordinate points, which are within a predetermined distance from the k-th subject spatial relative coordinate point or (ii) on a magnetic flux density table corresponding to the (k+1)-th subject spatial relative coordinate point by referring to each of (k+1)-th specific magnetic flux density tables corresponding to at least two or more (k+1)-th specific coordinate points, which are within a predetermined distance from the (k+1)-th subject spatial relative coordinate point.
